(19) United States
(12) Patent Application Publication
(10) Pub. No.: US 2016/0081895 A1
(43) Pub. Date: Mar. 24, 2016

(54) NOVEL FORMULATIONS

(71) Applicant: Stiefel Laboratories, Inc., Wilmington, DE (US)

(72) Inventors: Russell Phillip ELLIOTT, Belmont, CA (US); Connie Baozhen LIN, Research Triangle Park, NC (US); Harish MAHALINGAM, Parsippany, NJ (US); Swati MODI, Research Triangle Park, NC (US)

(21) Appl. No.: 14/855,482

(22) Filed: Sep. 16, 2015

Related U.S. Application Data
(60) Provisional application No. 62/166,906, filed on May 27, 2015.
(60) Provisional application No. 62/057,760, filed on Sep. 30, 2014.
(60) Provisional application No. 62/051,988, filed on Sep. 18, 2014.

(51) Int. Cl. A61K 8/35; A61K 8/37; A61K 8/362; A61Q 19/00; A61K 8/42; A61K 8/97; A61Q 17/04

(52) U.S. Cl. CPC A61K 8/35; A61K 8/97; A61K 8/37; A61Q 17/04; A61Q 19/007; A61Q 19/001; A61K 8/42; A61K 8/362; A61K 2800/524; A61K 2800/52; A61K 2800/596; A61K 2800/74; A61K 2800/26

(57) ABSTRACT

The invention provides for a topical o/w emulsion having moisturizing, and protecting, repairing or restoring the skin lipid barrier of the lips of a mammal, and is a topical oil-in-water emulsion composition comprising:
(a) a discontinuous oil phase;
(b) a continuous aqueous phase comprising water and glycerin, wherein glycerin is present in an amount greater than about 12% w/w;
(c) a thickening agent; and
(d) at least one lamellar membrane structure; and
   wherein the composition is a lip protectant composition.

### FIELD OF THE INVENTION

The present invention relates to a novel moisturizing and barrier repair and/or restoration lip protectant composition comprising high levels of glycerin.

### BACKGROUND OF THE INVENTION

Unprotected skin is susceptible to dehydration and becoming irritated from exposure to the elements. This is especially true for the tips, which have been found to be even more vulnerable to water loss than typical skin. In this regard, the lips have a thinner stratum corneum and also contain lesser amounts of lipids than skin on other parts of the body. When the lipid barrier is depleted or is inadequate, the lips dry out becoming irritated and prone to cracking. Lips also contain less melanin than other areas of skin, and thus are at risk of sunburn and UV damage. Accordingly, effective lip protectant compositions are highly desirable.

Many products have been introduced into the market to keep the lips in a moisturized and smooth condition, and protect them from damage. These products typically contain waxes and/or oils that mitigate the amount of moisture that is lost, known as trans-epidermal water loss. Some products may additionally contain emollients, humectants and healing agents.

A conventional lipstick includes five basic components: waxes, emollients, functional ingredients, stabilizers and colorants. Waxes and emollients tend to make up the base to which the other non-aqueous ingredients are added. A lipstick base, as such, tends to be anhydrous and simply minimizes the amount of trans-epidermal water loss, rather than replace any lost moisture.

Moisturizing compositions are typically oil-in-water emulsions and usually contain thickeners and/or conventional emulsifiers to stabilize the emulsion. Such compositions have a relatively high water content and so are able to replace moisture lost from the stratum comeum. They also typically contain one or more humectants to help retain moisture. However, while moisturizing compositions temporarily decrease visible scaling and roughness of the skin, they may offer little improvement to the integrity of the stratum corneum barrier. In fact, common moisturizing compositions which contain conventional emulsifiers can actually cause disruptions to the barrier function of the skin. Thus, a composition with high water content may suggest that the product provides good moisturization, but it will not necessarily maintain, protect or restore the barrier function of the skin.

Accordingly, an effective topical composition that will maintain, protect and restore good barrier function to the lips is needed.

U.S. Pat. No. 5,643,899, Elias et al., discloses compositions directed specifically to treatment of epidermal barrier disorders such as hyperproliferative cutaneous diseases, papulosquamous diseases, and eczematous diseases. The disclosed compositions contain various combinations of essential lipids that include cholesterol and a ceramide, particularly acylceramide. The compositions, while described for repair of the epidermal barrier function, do not discuss application to the lips.

U.S. Pat. No. 5,508,034, Bernstein et al., discloses compositions containing various lipids naturally found in the stratum corneum as essential components for the treatment of dry skin disorders. These compositions must contain a fatty acid, cholesterol, and a phospholipid or a glycolipid. The compositions, while described for repair of the epidermal barrier function, do not discuss application to the lips.

U.S. Pat. No. 6,663,853, Singh, discloses compositions as lip care moisturizing products which comprise fatty acid esters, a wax, an emulsifier and 1.0% unilamellar liposomes in a water-in-oil emulsion. The liposomes contain a mixture of water and glycerin. The emulsions are stated to preferably contain squalane and panthenol.

Accordingly, an object of the present invention is to provide a topical composition that is effective in moisturizing the lips an optimally minimizing transepidermal water loss, while also protecting and repairing barrier function. A further object of the present invention is to provide a topical composition which is convenient, easily applied to the lips and cosmetically elegant.

### SUMMARY OF THE INVENTION

One embodiment of the disclosure is a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent; and
(d) at least one lamellar membrane structure; and
   wherein the composition is a lip protectant composition.

In one embodiment, the glycerin in the aqueous phase is present in an amount from about 12% to about 40% by weight, based on the total weight of the composition. In another embodiment, the glycerin in the aqueous phase is present in an amount from about 20% to about 30% by weight, based on the total weight of the composition. In yet another embodiment, the glycerin in the aqueous phase is present in an amount from about 20% to about 25% by weight, based on the total weight of the composition.

Another embodiment of the disclosure is a method for moisturizing, and protecting, repairing, or restoring the skin lipid barrier of the lips of a mammal, the method comprising applying to the lips of the mammal in need thereof an effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent; and
(d) at least one lamellar membrane structure; and
   wherein the composition is a lip protectant composition.

In one embodiment, the glycerin in the aqueous phase is present in an amount from about 12% to about 40% by weight, based on the total weight of the composition. In another embodiment, the glycerin in the aqueous phase is present in an amount from about 20% to about 30% by weight, based on the total weight of the composition. In yet another embodiment, the glycerin in the aqueous phase is present in an amount from about 20% to about 25% by weight, based on the total weight of the composition.

Another embodiment of the disclosure is a method of protecting the lips of a mammal with broad spectrum protection of a UVA sunscreen and a UVB sunscreen, and enriched in UVA protection, the method comprising applying to the lips of the mammal in need thereof an effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure;
(e) at least one UVA sunscreen and at least one UVB sunscreen; and wherein the UVA:SPF protection ratio is about 1:1; and
   wherein the composition is a lip protectant composition.

In one embodiment, the glycerin in the aqueous phase is present in an amount from about 12% to about 40% by weight, based on the total weight of the composition. In another embodiment, the glycerin in the aqueous phase is present in an amount from about 20% to about 30% by weight, based on the total weight of the composition. In yet another embodiment, the glycerin in the aqueous phase is present in an amount from about 20% to about 25% by weight, based on the total weight of the composition.

In one embodiment, the 1:1 protection ratio helps to protect against UVA photodegradation of pheomelanin. In an embodiment, the UVA sunscreen is Avobenzone. In another embodiment, the UVB sunscreen is Ethylhexyl Salicylate (Octisalate). In yet another embodiment, the composition further comprises a sunfilter stabilizer. In a further embodiment, the sunfilter stabilizer is Diethylhexyl Syringylidene Malonate.

Another embodiment of the disclosure is a topical oil-in-water emulsion composition comprising:
(a) a discontinuous oil phase;
(b) a continuous aqueous phase;
(c) a thickening agent;
(d) at least one lamellar membrane structure, comprising a phospholipid, water, and at least one of rice bran oil and rice bran wax; and
(e) optionally at least one dermatologically acceptable excipient.

Another embodiment of the disclosure is a novel lamellar membrane structure concentrate composition which comprises at least one lamellar membrane structure, comprising a phospholipid, water, and at least one of rice bran oil and rice bran wax; and optionally at least one of a lipid, squalane, a phytosterol, cholesterol or cholesterol derivative, a ceramide, and a triglyceride.

Another embodiment of the disclosure is a method of protecting the lips of a mammal against reactivation of herpes simplex virus, the method comprising applying to the lips of the mammal in need thereof an effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure; and
(e) at least one UVA sunscreen and at least one UVB sunscreen; and wherein the UVA/SPF protection ratio is about 1:1; and
   wherein the composition is a lip protectant composition.

Yet another embodiment of the disclosure is a method of protecting the lips of a mammal against a reoccurrence of cold sores, the method comprising applying to the lips of the mammal in need thereof an effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure; and
(e) at least one UVA sunscreen and at least one UVB sunscreen; and wherein the UVA/SPF protection ratio is about 1:1; and
   wherein the composition is a lip protectant composition.

An embodiment of the disclosure is a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent; and
(d) at least one lamellar membrane structure; and
   wherein the composition is a lip protectant composition, for use in moisturizing, and protecting, repairing, or restoring the skin lipid barrier of the lips of a mammal.

Another embodiment of the disclosure is a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure; and
(e) at least one UVA sunscreen and at least one UVB sunscreen; and wherein the UVA:SPF protection ratio is about 1:1; and
   wherein the composition is a lip protectant composition, for use in protecting the lips of a mammal with broad spectrum protection of a UVA sunscreen and a UVB sunscreen, and enriched in UVA protection.

Yet another embodiment of the disclosure is a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure; and
(e) at least one UVA sunscreen and at least one UVB sunscreen; and wherein the UVA/SPF protection ratio is about 1:1; and
   wherein the composition is a lip protectant composition, for use in protecting the lips of a mammal against reactivation of herpes simplex virus.

A further embodiment of the disclosure is a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure; and
(e) at least one UVA sunscreen and at least one UVB sunscreen; and wherein the UVA/SPF protection ratio is about 1:1; and
   wherein the composition is a lip protectant composition, for use in protecting lips against a reoccurrence of cold sores.

### BRIEF DESCRIPTION OF THE DRAWINGS

FIG. 1 illustrates the emulsion ultrastructure of Example 1B using cryo-TEM (transmission electron microscopy).

FIG. 2 illustrates a Lip balm with UV filters inhibiting UVB-induced DNA damage (CPD, pink staining) and apoptosis (CC3, brown staining) in EpiDerm.

FIG. 3 illustrates the results of a Lip balm with UV filter inhibiting UVB-induced pro-inflammatory mediators in EpiDerm.

FIG. 4 illustrates a Lip balm with UV filters inhibiting UVB-induced DNA damage (CPD, pink staining) and apoptosis (CC3, brown staining) in EpiGingival.

FIG. 5 illustrates a Lip balm with UV filters inhibiting UVB-induced pro-inflammatory mediators in EpiGingival.

FIG. 6 illustrates a Lip balm with UV filters inhibiting UVA-induced DNA damage and apoptosis in EpiDerm^{FT}.

FIG. 7 illustrates a Lip balm with UV filters inhibiting UVA-induced pro-inflammatory mediators and PGE₂ in EpiDerm^{FT}.

FIG. 8 illustrates tissues with controls, placebo's and the protective activities of Lip balms with UV filters in EpiGingival.

FIG. 9 graphically illustrates tissues with controls, placebo's and the protective activities of Lip balms with UV filters in EpiGingival.

### DETAILED DESCRIPTION OF THE INVENTION

In one embodiment, the invention provides a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent; and
(d) at least one lamellar membrane structure; and
   wherein the composition is a lip protectant composition.

In one embodiment, the glycerin in the aqueous phase is present in an amount from about 12% to about 40% by weight, based on the total weight of the composition. In another embodiment, the glycerin in the aqueous phase is present in an amount from about 20% to about 30% by weight, based on the total weight of the composition. In yet another embodiment, the glycerin in the aqueous phase is present in an amount from about 20% to about 25% by weight, based on the total weight of the composition.

In one embodiment, the composition is a lipstick. In another embodiment, the composition is a lip balm. In yet another embodiment, the composition is a stick lip balm. In a further embodiment, the composition is a lip cream. In yet a further embodiment, the composition is a lip balm, a lip cream, or a stick lip balm.

### Oil Phase

The compositions of this disclosure comprise a discontinuous oil phase. The discontinuous oil phase is dispersed throughout the continuous aqueous phase.

In an embodiment, the discontinuous oil phase comprises at least one oil and/or fat. In one embodiment, the oil and/or fat is a mixture of two or more oils and/or fats. Exemplary oils and fats include, but are not limited to, fatty acids, fatty alcohols, esters, esters of glycerin, waxes, sterols, essential oils, vegetable oils and edible oils, and mixtures thereof.

Exemplary fatty acids include, but are not limited to, isostearic acid, linoleic acid, linolenic acid, oleic acid, myristic acid, ricinoleic acid, columbinic acid, arachidic acid, arachidonic acid, lignoceric acid, nervonic acid, eicosapentanoic acid, palmitic acid, stearic acid and behenic acid, and mixtures thereof.

The fatty acid can be introduced into the present compositions from a variety of sources. In an embodiment, the fatty acid is provided in the composition as an oil or wax. Examples of oils useful in this regard include, but are not limited to, rice bran oil, flaxseed oil, hempseed oil, pumpkin seed oil, canola oil, soybean oil, wheat germ oil, olive oil, grapeseed oil, borage oil, evening primrose oil, black currant seed oil, chestnut oil, corn oil, safflower oil, sunflower oil, sunflower seed oil, cottonseed oil, peanut oil, sesame oil and olus (vegetable) oil, and mixtures thereof. An exemplary wax useful in this regard are the natural waxes, exemplified by rice bran wax.

In one embodiment, the source of fatty acids is shea butter, also known as Butyrospermum parkii. Shea butter comprises five principal fatty acids, namely palmitic acid, stearic acid, oleic acid, linoleic acid and arachidic acid. Shea butter also comprises phytosterols.

Exemplary fatty alcohols include, but are not limited to, behenyl alcohol, isostearyl alcohol, caprylyl alcohol, decyl alcohol, lauryl alcohol, myristyl alcohol, lanolin alcohol, arachidyl alcohol, oleyl alcohol, palm alcohol, isocetyl alcohol, cetyl alcohol, stearyl alcohol and cetearyl alcohol, and mixtures thereof. In one embodiment, the fatty alcohol is behenyl alcohol.

Exemplary esters include, but are not limited to, coco-caprylate/caprate, diethyl sebacate, diisopropyl adipate, diisopropyl dilinoleate, ethyl oleate, ethylhexyl hydroxystearate, glycol distearate, glycol stearate, hydroxyoctacosanyl hydroxystearate, isopropyl isostearate, isostearyl isostearate, isopropyl myristate, isopropyl palmitate, isopropyl stearate, methyl glucose sesquistearate, methyl laurate, methyl salicylate, methyl stearate, myristyl lactate, octyl salicylate, oleyl oleate, PPG-20 methyl glucose ether distearate, propylene glycol diacetate, propylene glycol dicaprylate, propylene glycol monolaurate, propylene glycol monopalmitostearate, propylene glycol ricinoleate and sucrose distearate, and mixtures thereof.

Exemplary esters of glycerin include, but are not limited to, caprylic/capric triglycerides, caprylic/capric/succinic triglyceride, cocoglycerides, glyceryl citrate, glyceryl isostearate, glyceryl laurate, glyceryl monostearate, glyceryl oleate, glyceryl palmitate, glyceryl ricinoleate, glyceryl stearate, mono and diglyceride, PEG-12 glyceryl laurate, PEG-120 glyceryl stearate, polyglyceryl-3 oleate, polyoxyl glyceryl stearate, glycerol monocaprin, glycerol monolaurin, tallow glycerides and medium chain triglycerides, and mixtures thereof. In one embodiment, triglycerides isolated from palm oil are preferred. In one embodiment, the monoglycerol derivative is a C8-C16 derivative. In another the monoglycerol derivative is a C8-C12 ester.

Waxes typically serve as structurants for stick lip balms permitting the suck to be extended and retracted in use while maintaining the stick form. Suitable waxes for stick compositions include animal waxes, plant waxes, mineral waxes, silicone waxes, synthetic waxes and petroleum waxes. Exemplary waxes include, but are not limited to, rice bran wax, carnauba wax, paraffin wax, white wax, candelilla wax, beeswax, jojoba wax and ozokerite, and mixtures thereof.

Exemplary sterols include, but are not limited to, Brassica Campestris sterols, C₁₀-C₃₀ cholesterol/lanosterol esters, canola sterols, cholesterol, cholesterols, glycine soja sterols, PEG-20 phytosterol and phytosterols, and mixtures thereof.

Exemplary essential oils include, but are not limited to, primrose oil, rose oil, eucalyptus oil, borage oil, bergamot oil, chamomile oil, citronella oil, lavender oil, peppermint oil, pine oil, pine needle oil, spearmint oil, tea tree oil and wintergreen oil, and mixtures thereof.

Exemplary vegetable oils include, but are not limited to, olus (vegetable) oil, almond oil, aniseed oil, canola oil, castor oil, coconut oil, corn oil, avocado oil, cottonseed oil, olive oil, palm kernel oil, peanut oil, sunflower oil, safflower oil and soybean oil, and mixtures thereof.

Exemplary edible oils include, but are not limited to, cinnamon oil, clove oil, lemon oil and peppermint oil, and mixtures thereof.

Suitably, the discontinuous oil phase is present in an amount from about 5% to about 70% by weight, based on the total weight of the composition.

### Aqueous Phase

The compositions of the invention comprise a continuous aqueous phase. The aqueous phase comprises water. Suitably, any additional components such as glycerin and any other water soluble excipients will be dissolved in this aqueous phase. Suitably, the continuous aqueous phase is present in an amount from about 10% to about 90% by weight, based on the total weight of the composition. In another embodiment the continuous aqueous phase is present in an amount from about 25% to about 90% by weight, based on the total weight of the composition. In an embodiment, the continuous aqueous phase is present in an amount from about 25% to about 75% by weight, based on the total weight of the composition. In another embodiment, the continuous aqueous phase is present in an amount from about 25% to about 70% by weight, based on the total weight of the composition.

In an embodiment, the continuous aqueous phase comprises water in an amount from about 13% to about 60% by weight, in another embodiment from about 15% to about 40% by weight, and in another embodiment from about 15% to about 35% by weight, based on the total weight of the composition. In another embodiment from about 20% to about 40% by weight, based on the total weight of the composition.

In an embodiment, the continuous aqueous phase comprises glycerin present in an amount from about 12% to about 40% by weight, based on the total weight of the composition. In another embodiment, the continuous aqueous phase comprises glycerin in an amount from about 18% to about 30% by weight, based on the total weight of the composition. In another embodiment, the continuous aqueous phase comprises glycerin in an amount from about 20% to about 40% by weight, based on the total weight of the composition. In another embodiment, the continuous aqueous phase comprises glycerin in an amount from about 20% to about 30% by weight, based on the total weight of the composition. In yet another embodiment, the continuous aqueous phase comprises glycerin in an amount from about 20% to about 25% by weight, based on the total weight of the composition. In another embodiment, the continuous aqueous phase comprises glycerin in an amount of about 20%, 21%, 22%, 23%, 24 or 25% by weight, based on the total weight of the composition.

In one embodiment, the continuous aqueous phase may also include a sugar alcohol, such as glucose, sorbitol, mannitol, maltitol, galactitol, erythritol, xylitol, inositol, lactitol, and mixtures thereof. In one embodiment, the sugar alcohol is glucose. The sugar alcohol may be present in an amount from about 1% to about 20% by weight, based on the total weight of the composition. In one embodiment of the disclosure, the sugar alcohol is present in an amount from about 10% to about 15% by weight, based on the total weight of the composition. In a more preferred embodiment, the sugar alcohol is present in an amount of about 10%, 11%, 12%, 13%, 14% or 15% by weight, based on the total weight of the composition.

The continuous aqueous phase may further comprise other water miscible components, such as for example, humectants, pH adjusting agents antioxidants, and SPF boosters.

### Thickening Agent

The compositions of the invention comprise a thickening agent or rheology modifier. In an embodiment, the thickening agent is a mixture of two or more thickening agents.

The function of the thickening agent is to stabilize the discontinuous oil phase of the composition. The thickening agent may also provide hardness and structural support useful in forming a stick composition, for example. Thickening agents may be water miscible which are used to thicken the aqueous portion of the emulsion composition. Other thickening agents are nonaqueous making them suitable for thickening the oil phase of the emulsion composition. Yet other thickening agents may act at the oil-water interface and thus lie at the interphase boundary.

Exemplary water miscible thickening agents include, but are not limited to, a cellulose derivative such as carboxymethylcellulose, hydroxyethylcellulose, hydroxypropylcellulose, hydroxypropyl methylcellulose; agar; carrageenan; curdlan; gelatin; gellan; β-glucan; tragacanth gum; guar gum; gum arabic; locust bean gum; pectin; starch; a carbomer, such as sodium carbomer; a xanthan derivative such as dehydroxanthan gum and xanthan gum; salts thereof, or a combination or mixture thereof.

Exemplary nonaqueous thickening agents include, but are not limited to, acrylate copolymers, VP/Eicosene copolymer, waxes, fatty alcohols and fatty acids, as described herein.

In an embodiment, the thickening agent is an acrylate copolymer, such as acrylates/C10-30 alkyl acrylate cross polymer.

In one embodiment, the thickening agent is xanthan gum. In another embodiment, the thickening agent is dehydroxanthan gum. In yet another embodiment, the thickening agent is a carbomer or a salt thereof, such as sodium carbomer. In a further embodiment, the thickening agent is hydroxyethylcellulose.

In one embodiment, the thickening agent is a fatty alcohol. Suitable fatty alcohols include, but are not limited to, behenyl alcohol, isostearyl alcohol, caprylyl alcohol, decyl alcohol, lauryl alcohol, myristyl alcohol, lanolin alcohol, arachidyl alcohol, oleyl alcohol, palm alcohol, isocetyl alcohol, cetyl alcohol, stearyl alcohol and cetearyl alcohol, and mixtures thereof.

In another embodiment, the thickening agent is a fatty acid. Suitable fatty acids include, but are not limited to, isostearic acid, linoleic acid, linolenic acid, oleic acid, myristic acid, ricinoleic acid, columbinic acid, arachidic acid, arachidonic acid, lignoceric acid, nervonic acid, eicosapentanoic acid, palmitic acid, stearic acid and behenic acid, and mixtures thereof.

In one embodiment, the thickening agent comprises a mixture of fatty alcohols, a cellulose derivative, a xanthan derivative, a non-aqueous agent, and a carbomer. In one embodiment, the thickening agent comprises behenyl alcohol, dehydroxanthan gum, VP/Eicosene copolymer, acrylates/C10-30 alkyl acrylate cross polymer and sodium carbomer.

Suitably, the thickening agent is present in an amount from about 0.5% to about 10% by weight, based on the total weight of the composition. In an embodiment, the thickening agent is present in an amount from about 1% to about 5% by weight, based on the total weight of the composition.

### Lamellar Membrane Structure

The compositions of the invention comprise at least one lamellar membrane structure, which is a planar lipid bilayer sheet. In another embodiment, the respective lamellar membrane structures form two or more stacked lamellar membrane structures. Two lamellar membrane structures stacked together, one on top of the other, is known as a double lamellar membrane structure.

In an embodiment, the at least one lamellar membrane structure comprises a phospholipid and water. In an embodiment, the phospholipid is lecithin. In one embodiment, the phospholipid is hydrogenated lecithin. In another embodiment, the phospholipid is phosphatidylcholine. In yet another embodiment, the phospholipid is hydrogenated phosphatidylcholine. In a further embodiment, the phospholipid is a mixture of phosphatidylcholine and hydrogenated phosphatidylcholine. One suitable source of hydrogenated lecithin is Phospholipon 90H®, available from Lipoid GmbH (Ludwigshafen, Germany).

As used herein, “phosphatidylcholine” (PC) is a class of phospholipids that incorporate choline as a headgroup. Purified phosphatidylcholine is produced commercially. Phosphatidylcholines may be from any source, such as soy or egg. Soy phosphatidylcholine is characterized by a proportion of linoleic acid up to 70% of the total fatty acids. Egg phosphatidylcholine contains 28-38% palmitic acid, 9-18% stearic acid, 25-37% oleic acid, 12-17% linoleic acid, about 0.5% linolenic acid and 1-7% arachidonic acid. The phospholipids herein may also include hydrogenated PC's, such as soy phosphatidylcholine which contains mainly stearic and palmitic acids, and semisynthetic compounds such as dipalmitoyl phosphatidylcholine and distearoyl phosphatidylcholine. By way of clarification, it is to be noted that the term phospholipid covers not only a single phospholipid but also a mixture of phospholipids, wherein the phospholipid or respectively the phospholipid mixture can be of natural or synthetic origin. It is likewise self-evident that the phospholipid can be hydrogenated, but that instead of this hydrogenated phospholipid a synthetic phospholipid can be used, e.g. in which the acyl radicals are all or predominantly saturated in the above sense.

In one embodiment of the disclosure, the hydrogenated phosphatidylcholine is at least 60% by weight hydrogenated phosphatidylcholine.

Suitably, the phospholipid is present in an amount from about 0.50% to about 95% by weight, based on the total weight of the composition. In an embodiment, the phospholipid is present in an amount from about 0.1% to about 95% by weight, based on the total weight of the composition. In another embodiment, the phospholipid is present in an amount from about 0.5% to about 15% by weight, based on the total weight of the composition. In another embodiment, the phospholipid is present in an amount from about 0.1% to about 15% by weight, based on the total weight of the composition. In another embodiment, the phospholipid is present in an amount from about 0.5% to about 7% by weight, based on the total weight of the composition. In yet another embodiment, the phospholipid is present in an amount from about 0.5% to about 1% by weight, based on the total weight of the composition.

In one embodiment, the at least one lamellar membrane structure comprises a phospholipid, water and a lipid. In an embodiment, the lamellar membrane structure comprises a phospholipid, water and a lipid, and optionally a polyvalent alcohol.

As used herein, “lipid” refers to an oil (as a liquid), a semi-solid (a butter) or a solid (wax) component.

Suitably, the lipid is an oil. Exemplary oils include, but are not limited to, fatty acids, a source of fatty acids, esters, esters of glycerin (including mono-, di- and tri-esters), sterols, essential oils, vegetable oils, edible oils, and mixtures thereof. In one embodiment, the oil is a fatty acid, a source of fatty acids, or an ester of glycerin, as described herein.

Exemplary fatty acids include, but are not limited to, isostearic acid, linoleic acid, linolenic acid, oleic acid, myristic acid, ricinoleic acid, columbinic acid, arachidic acid, arachidonic acid, lignoceric acid, nervonic acid, eicosapentanoic acid, palmitic acid, stearic acid, and behenic acid, and mixtures thereof.

The fatty acid can be introduced into the present compositions from a variety of sources. In an embodiment, the fatty acid is provided in the composition as an oil. Examples of oils useful in this regard include, but are not limited to, rice bran oil, flaxseed oil, hempseed oil, pumpkin seed oil, canola oil, soybean oil, wheat germ oil, olive oil, grape seed oil, borage oil, evening primrose oil, black currant seed oil, chestnut oil, corn oil, safflower oil, sunflower oil, sunflower seed oil, cottonseed oil, peanut oil, sesame oil and olus (vegetable) oil, and mixtures thereof.

In an embodiment, the source of fatty acids is olus (vegetable) oil, olive oil or rice bran oil. In another embodiment, the source of fatty acids is rice bran oil, rice bran wax, or a mixture of rice bran oil and rice bran wax.

Exemplary esters include, but are not limited to, coco-caprylate/caprate, diethyl sebacate, diisopropyl adipate, diisopropyl dilinoleate, ethyl oleate, ethylhexyl hydroxystearate, glycol distearate, glycol stearate, hydroxyoctacosanyl hydroxystearate, isopropyl isostearate, isostearyl isostearate, isopropyl myristate, isopropyl palmitate, isopropyl stearate, methyl glucose sesquistearate, methyl laurate, methyl salicylate, methyl stearate, myristyl lactate, octyl salicylate, oleyl oleate, PPG-20 methyl glucose ether distearate, propylene glycol diacetate, propylene glycol dicaprylate, propylene glycol monolaurate, propylene glycol monopalmitostearate, propylene glycol ricinoleate and sucrose distearate, and mixtures thereof.

Exemplary esters of glycerin include, but are not limited to, caprylic/capric triglycerides, caprylic/capric/succinic triglyceride, cocoglycerides, glyceryl citrate, glyceryl isostearate, glyceryl laurate, glyceryl monostearate, glyceryl oleate, glyceryl palmitate, glyceryl ricinoleate, glyceryl stearate, mono and diglyceride, PEG-12 glyceryl laurate, PEG-120 glyceryl stearate, polyglyceryl-3 oleate, polyoxyl glyceryl stearate, tallow glycerides and medium chain triglycerides, and mixtures thereof.

In one embodiment, the ester of glycerin is a mono-, di- or triglyceride, such as caprylic/capric triglyceride. In one embodiment, triglycerides isolated from palm oil are preferred. In another embodiment the monoglycerol derivative is a C8-C16 derivative. In another the monoglycerol derivative is a C8-C12 ester.

Exemplary essential oils include, but are not limited to, primrose oil, rose oil, eucalyptus oil, borage oil, bergamot oil, chamomile oil, citronella oil, lavender oil, peppermint oil, pine oil, pine needle oil, spearmint oil, tea tree oil and wintergreen oil, and mixtures thereof.

Exemplary vegetable oils include, but are not limited to, olus (vegetable) oil, almond oil, aniseed oil, canola oil, castor oil, coconut oil, corn oil, avocado oil, cottonseed oil, olive oil, palm kernel oil, peanut oil, sunflower oil, safflower oil and soybean oil, and mixtures thereof. One embodiment is the use of olive oil and/or vegetable oil.

Exemplary edible oils include, but are not limited to, cinnamon oil, clove oil, lemon oil and peppermint oil, and mixtures thereof.

In one embodiment, the lipid is a butter, such as shea butter, also known as Butyrospermum parkii. Shea butter comprises five principal fatty acids, namely palmitic acid, stearic acid, oleic acid, linoleic acid and arachidic acid. Shea butter also comprises phytosterols.

In one embodiment, the lipid is a wax. Suitable waxes include, but are not limited to, animal waxes, plant waxes, mineral waxes, silicone waxes, synthetic waxes and petroleum waxes, and mixtures thereof. Exemplary waxes also include, rice bran wax, carnauba wax, paraffin wax, white wax, candelilla wax, beeswax, jojoba wax and ozokerite, and mixtures thereof. In one embodiment, the wax is rice bran wax.

Suitably, the lipid is present in an amount from about 0.5% to about 2% by weight, based on the total weight of the composition. In an embodiment, the ratio of the lipid to the phospholipid is from about 0.5:1 to about 1:1.

In an embodiment, the at least one lamellar membrane structure comprises a phospholipid, water, and a phytosterol, or cholesterol or cholesterol derivative.

In another embodiment, the at least one lamellar membrane structure comprises a phospholipid, water and squalane.

In yet another embodiment, the at least one lamellar membrane structure comprises a phospholipid, water, and at least one of rice bran oil and rice bran wax.

In a further embodiment, the at least one lamellar membrane structure comprises a phospholipid, water, a lipid, and at least one of a phytosterol, squalane, rice bran oil and rice bran wax.

In yet a further embodiment, the at least one lamellar membrane structure further comprises a ceramide.

In an embodiment, the at least one lamellar membrane structure comprises a phospholipid, water, a lipid, at least one of a phytosterol, squalane, rice bran oil, rice bran wax, and a ceramide.

In another embodiment, the at least one lamellar membrane structure comprises a phospholipid, water, a lipid, and a phytosterol, and optionally at least one of squalane, rice bran oil, rice bran wax, pentylene glycol and/or hexylene glycol and a ceramide.

In yet another embodiment, the at least one lamellar membrane structure comprises a phospholipid, water, a phytosterol, squalane, rice bran oil and rice bran wax.

In a further embodiment, the at least one lamellar membrane structure comprises a phospholipid, water, and at least one of rice bran oil and rice bran wax; and optionally at least one of a lipid, squalane, a phytosterol, cholesterol or cholesterol derivative, a ceramide, or a triglyceride.

Many of the lipids used in the present compositions are the same or similar to the lipids found in human stratum corneum.

Suitably, the at least one lamellar membrane structure is present in an amount from about 0.5% to about 5% by weight, based on the total weight of the composition.

### Phytosterols/Cholesterol/Cholesterol Derivative

The term “phytosterol” refers to plant sterols and plant stanols. Plant sterols are naturally occurring cholesterol-like molecules found in all plants, with the highest concentrations occurring in vegetable oils. Plant stanols are hydrogenation compounds of the respective plant sterols.

Phytosterols are natural components of common vegetable oils. Exemplary sources of phytosterols useful in this regard include, but are not limited to, shea butter, vegetable oil, tall oil, sesame oil, sunflower oil, sunflower seed oil, rice bran oil, cranberry seed oil, pumpkin seed oil, avocado wax, and mixtures thereof. In one particular embodiment, the source of phytosterols is shea butter or soy.

The majority of the previously known compositions contain cholesterol, or an animal-based sterol, rather than a phytosterol. The use of a phytosterol in embodiments of the present invention, rather than cholesterol, is advantageous.

In this regard, phytosterols are typically incorporated in the basal membrane of the skin and can pass to the skin surface through the differentiation of skin cells. Accordingly, phytosterols provide an improved caring and protecting effect. The topical application of phytosterols also usually leads to an increased skin moisture level and to increased lipid content. This improves the desquamation behavior of the skin and reduces erythemas which may be present. R. Wachter, Parf. Kosm., Vol. 75, p. 755 (1994) and R. Wachter, Cosm. Toil., Vol. 110, p. 72 (1995), each of which are incorporated herein by reference in their entirety, further demonstrate these advantageous properties of phytosterols.

As used herein, “cholesterol derivative” is any suitable dermatologically acceptable sterol variation of cholesterol.

Suitably, the phytosterol, source of phytosterols, cholesterol, or cholesterol derivative is present in the at least one lamellar membrane structure in an amount from about 0.05% to about 2% by weight, based on the total weight of the composition. It is understood that these sterols are considered a lipid component.

### Squalane

Squalane helps enhance the skin's natural barrier function, protect the skin against the elements, and boost the skin's ability to retain moisture. Squalane is a derivative of squalene, which is a component of human stratum corneum.

Squalane is available in purified form (see e.g. Fitoderm® available from BASF) and may be used in the compositions in its purified form. Alternatively, an oil which is rich in squalane may be used.

Exemplary sources of squalane useful in the present compositions include, but are not limited to, shark liver oil, olive oil, palm oil, wheat germ oil, amaranth oil, rice bran oil and sugar cane. It is understood that squalane from these sources of oils is considered a lipid component. In one embodiment, squalane isolated from olive oil is preferred. Suitably, the squalane or squalene is present in the at least one lamellar membrane structure in an amount from about 0.05% to about 2% by weight, based on the total weight of the composition.

### Ceramides

Ceramides are a family of waxy lipid molecules composed of sphingosine and a fatty acid. They contain an acyl linkage and the chain length of the most abundant chain is C₂₄-C₂₆ with a small fraction having an acyl chain length of C₁₆-C₁₈. Ceramides are found extensively in the stratum corneum. Ceramides are commercially available from major chemical suppliers such as Evonik or Sigma Chemical Company, St. Louis, Mo., U.S.A.

Exemplary ceramides useful in the present compositions include, but are not limited to, ceramide-1, -2, -3, -4, -5, -6 or -7, and mixtures thereof. Other ceramides known to those of skill in the art as useful in topical compositions are further contemplated as useful in the present compositions, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, January 1996, the contents of which are hereby incorporated by reference in their entirety.

In one embodiment, the ceramide is ceramide-3.

Suitably, the ceramide is present in the at least one lamellar membrane structure in an amount from about 0.001% to about 1% by weight, based on the total weight of the composition.

Ceramides, acylceramides and glucosylceramides are all members of the “sphingoid” or “spingolipids” class. As noted above, these are compounds which have a backbone of sphingosine or a closely related structure to which either fatty acids or ω-esterified fatty acids are linked through an amide linkage at the amino group of the sphingosine structure and in the case of a glucosylceramide, those to which saccharide moieties are linked to the terminal hydroxyl of the sphingosine structure through a glycosidic bond.

One embodiment of the disclosure is a phytosterol, cholesterol or cholesterol derivative in combination with a sphingoid or sphingolipid. More preferably, the sphingoid or sphingolipid is a ceramide and/or is a phytosphingosine.

The at least one lamellar membrane structure can be prepared prior to formulating final compositions of the present invention. In one embodiment, the lamellar membrane structure may also be referred to as a dermal membrane structure, e.g. a DMS® concentrate (also referred to herein as Probiol™) prepared in accordance with the teachings of several patents and patent application as disclosed in detail in Albrecht et al., U.S. Pat. No. 7,001,604; Albrecht et al., US 2011/0027327; and Albrecht et al., WO 2007/112712 which disclosures are incorporated by reference in part.

In an embodiment, the phospholipid in the DMS® concentrate is hydrogenated lecithin, and the concentrate further comprises water and a lipid.

In an embodiment, the present invention is also directed to a novel lamellar membrane structure composition comprising a phospholipid, water, and at least one of rice bran oil and rice bran wax.

In another embodiment, the lamellar membrane structure composition comprises a phospholipid, water, and rice bran oil and rice bran wax.

Rice bran oil is also known as Oryza Sativa bran oil, and rice bran wax is also known as Oryza Sativa Cera. Rice bran oil has a composition similar to peanut oil, with 38% monounsaturated, 37% polyunsaturated and 25% saturated fatty acids. More specifically, the fatty acid composition of rice bran oil is:

TABLE 1
Fatty acid composition of rice bran oil

| Fatty acid | Percentage |
|---|---|
| C14:0 Myristic acid | 0.6% |
| C16:0 Palmitic acid | 21.5% |
| C18:0 Stearic acid | 2.9% |
| C18:1 Oleic acid | 38.4% |
| C18:2 Linoleic acid | 34.4% |
| C18:3 α-Linolenic acid | 2.2% |

Rice Bran Wax is the vegetable wax extracted from the bran oil of rice. It contains C₁₆-C₃₀ fatty acids.

In one embodiment, the lamellar membrane structure composition comprises hydrogenated lecithin, shea butter, squalane, pentylene glycol, glycerin, ceramide-3, rice bran oil, rice bran wax, phytosphingosine, palmitidyl monoethanolamide (MEA) or referred to as (PMEA), and water.

In one embodiment, the lamellar membrane structure composition comprises hydrogenated phosphatidylcholine, shea butter, squalane, pentylene glycol and/or hexylene glyco, glycerin, ceramide-3, rice bran oil, rice bran wax, phytosphingosine, palmitidyl monoethanolamide (MEA) or referred to as (PMEA), and water.

Kuhs GmBH has provided commercial information on various lamellar concentrates under the DMS® Concentrate line as DMS® 03007, 03015, 03016, 03017, 03020 and 03031 which are included for use within the invention herein.

TABLE 2

| Probiol No. | Lipids | Preservative | Colour | INCI |
|---|---|---|---|---|
| N 03007 | Caprylic/Capric Triglycerides | Alcohol | White | Aqua & Alcohol & |
| | Hydrogenated Lecithin | | | Caprylic/Capric |
| | | | | Triglycerides & |
| | | | | Hydrogenated Lecithin |
| N 03015 | Caprylic/Capric Triglycerides | Alcohol | White | Aqua & Alcohol & |
| | Shea Butter | | | Caprylic/Capric |
| | Squalane | | | Triglycerides & |
| | Ceramide 3 | | | Hydrogenated Lecithin & |
| | Hydrogenated Lecithin | | | Butyrospermum Parkii & |
| | | | | Squalane & Ceramide 3 |
| N 03017 | Caprylic/Capric Triglycerides | Alcohol | White | Aqua & Alcohol & |
| | Shea Butter | Propylene | | Caprylic/Capric |
| | Squalane | Glycol | | Triglycerides & |
| | Ceramide 3 | | | Hydrogenated Lecithin & |
| | Hydrogenated Lecithin | | | Propylene Glycol & |
| | | | | Butyrospermum Parkii & |
| | | | | Squalane & Ceramide 3 |
| N 03020 | Caprylic/Capric Triglycerides | Alcohol | White | Aqua & Alcohol & |
| | Hydrogenated Lecithin | Propylene | | Caprylic/Capric |
| | | Glycol | | Triglycerides & |
| | | | | Hydrogenated Lecithin & |
| | | | | Propylene Glycol |
| N 03031 | Caprylic/Capric Triglycerides | Pentylene | White | Aqua & Hydrogenated |
| | Shea Butter | Glycol | | Lecithin & |
| | Squalane | | | Caprylic/Capric |
| | Ceramide 3 | | | Triglycerides & |
| | Hydrogenated Lecithin | | | Pentylene Glycol & |
| | | | | Butyrospermum Parkii & |
| | | | | Glycerin & Squalane & |
| | | | | Ceramide 3 |

Suitably, the lamellar membrane structure as a concentrate can represent a phase in the final composition of about 5% to about 90% by weight, based on the total weight of the final composition. In one embodiment, the concentrate is present in an amount from about 10% to about 50% by weight, based on the total weight of the composition. In another embodiment, the lamellar membrane structure as a concentrate is present in an amount from about 10% to about 30% by weight, based on the total weight of the composition. In yet another embodiment, the lamellar membrane structure as a concentrate is present in an amount of about 15% by weight, based on the total weight of the composition.

In another embodiment of the disclosure, the lamellar membrane structure may further comprise at least one alcohol, in particular a polyvalent alcohol. Suitable polyvalent alcohols include, but are not limited to, pentylene glycol, hexylene glycol, caprylyl glycol, phenylethyl alcohol, decylene glycol, glycerin or mixtures thereof. In one embodiment, the lamellar membrane structure comprises glycerin. In another embodiment, the lamellar membrane structure comprises pentylene glycol. In another embodiment, the lamellar membrane structure comprises pentylene glycol and/or hexylene glycol and glycerin.

### Dermatologically Acceptable Excipients

The compositions of the invention may further comprise at least one dermatologically acceptable excipient.

In an embodiment, the dermatologically acceptable excipient is selected from the group consisting of an antioxidant, a chelating agent, a preservative, a colorant, a sensate, a moisturizer, a humectant, a lip conditioning agent and a pH adjusting agent, and mixtures thereof.

In an embodiment, the compositions of the invention are free or substantially free of a conventional emulsifier.

### Antioxidant

The compositions of the invention may further comprise an antioxidant. In an embodiment, the antioxidant is a mixture of two or more antioxidants.

Antioxidants may protect the composition from oxidation (e.g. becoming rancid) and/or provide lip conditioning benefits upon application to the lips. Tocopherol, tocopheryl acetate, some botanical butters, niacinamide, pterostilbene (trans-3,5-dimethoxy-4-hydroxystilbene) magnolol, and green tea extracts, alone or in combination thereof are exemplary natural product antioxidants suitable for use in the compositions. Other suitable antioxidants include ascorbic acid and esters thereof such as ascorbyl palmitate, butylated hydroxytoluene (BHT), butylated hydroxyanisole (BHA), propyl gallate, vitamin E TPGS, ethyl ferulate, ferulic acid, resveratrol, 2,2-dimethyl chroman (Lipochroman®), singapine, tetrahydrocurcumin or other curcumin derivatives, hydroxytyrosol, Bis-Ethylhexyl Hydroxydimethoxy Benzylmalonate (Ronacare AP®), dimethylmethoxy chromanyl palmitate (Chromabright®) or a combination or mixture thereof. It is recognized that a combination or mixture of all of these antioxidants is also suitable for use herein. In one embodiment, the antioxidant is tocopherol, or a mixture of tocopherol and ascorbyl palmitate. In another embodiment, the antioxidant is niacinamide.

Suitably, the antioxidant is present in an amount from about 0.001% to about 1% by weight, based on the total weight of the composition.

### Chelating Agents

The compositions of the invention may further comprise a chelating agent. In an embodiment, the chelating agent is a mixture of two or more chelating agents.

Exemplary chelating agents include, but are not limited to, citric acid, glucuronic acid, sodium hexametaphosphate, zinc hexametaphosphate, ethylenediamine tetraacetic acid (EDTA), ethylenediamine disuccinic acid (EDDS), phosphorates, salts thereof, or a combination or mixture thereof.

In one embodiment, the chelating agent is EDTA or a salt thereof, such as potassium, sodium or calcium salts of EDTA. In another embodiment, the chelating agent is ethylenediamine succinic acid or a salt thereof, such as potassium, sodium or calcium salts. In one particular embodiment, the chelating agent is trisodium ethylenediamine disuccinate.

Suitably, the chelating agent is present in an amount from about 0.1% to about 1% by weight, based on the total weight of the composition.

### Preservative

The compositions of the invention may further comprise a preservative. In an embodiment, the preservative is a mixture of two or more preservatives.

Exemplary preservatives include, but are not limited to, benzyl alcohol, diazolidinyl urea, methyl paraben, ethyl paraben, propyl paraben, butyl paraben, phenoxyethanol, sorbic acid, benzoic acid, salts thereof, or a combination or mixture thereof.

Suitably, the preservative is present in an amount from about 0.01% to about 2% by weight. In an alternative embodiment, the compositions of the invention are free of conventional preservatives.

In an embodiment, the preservative is a combination of non conventional preservatives, such as a combination of capryloyl glycine and a glycol. Suitable glycols include, but are not limited to, caprylyl glycol and/or pentylene glycol. Suitably, these preservatives are present in an amount from about 0.5% to about 5% by weight, based on the total weight of the composition. In one embodiment, the capryloyl glycine is present in an amount from about 0.5% to about 2% by weight and the glycol can be added in an amount up to 5% by weight, based on the total weight of the composition. Suitably, the preservative is a combination of at least capryloyl glycine and caprylyl glycol in an amount from about 0.5% to about 2% by weight, based on the total weight of the composition.

### Colorant

The compositions of the invention may further comprise a colorant that imparts color to the composition and/or lips. For a lip balm, the colorant should not be of an amount, particle size, and/or matrix that permits transfer of colorant to the lips during application. For a lipstick, a colorant that transfers and imparts color to the lips should be used. Colorants include, for example, natural colorants such as plant extracts, natural minerals, carmine, synthesized and/or processed colorant materials such as iron oxides, synthetic dyes, organic compounds, lake colorants, and FDA certified colorants for use on the lips. The above list is not an exhaustive list of colorants and those of skill in the art may consider the use of other colorants. Formulations of colorants are commercially available. An example of a commercially available colorant contains caprylic/capric triglycerides (59.5%), titanium dioxide (39.6%), castor oil phosphate (0.5%) and triethoxycaprylyisilane (0.4%). The use of a colorant containing titanium dioxide can affect the stability of some sunscreens such as Avobenzone. It has been observed that colorants containing coated titanium dioxide can enhance the stability of Avobenzone. Optionally, in some embodiments, it may be desirable to include a color enhancer such as, for example, a pearlescent material.

### Sensate

The compositions of the invention may further comprise a sensate. A sensate is a composition that initiates a sensory perception such as heating or cooling, for example, when contacted with the skin and/or lips. Exemplary sensates include, but are not limited to, mint extracts, cinnamon extract and capsaicin. Preferred sensates are derived from natural sources. However, synthetic sensates are within the scope of this invention. Sensates typically have high potency and accordingly may yield significant impact at low levels. Suitably, the sensate is present in an amount from about 0.05% to about 5% by weight, based on the total weight of the composition.

### Moisturizer

The compositions of the invention may further comprise a moisturizer. Exemplary moisturizers useful in the present compositions include, but are not limited to, pentylene glycol, hexylene glycol, butylene glycol, polyethylene glycol, sodium pyrrolidone carboxylate, α-hydroxy acids, β-hydroxy acids, polyhydric alcohols, ethoxylated and propoxylated polyols, polyols, polysaccharides, panthenol, hexylene glycol, propylene glycol, dipropylene glycol and sorbitol, and mixtures thereof.

Suitably, the moisturizer is present in an amount from about 0.5% to about 10% by weight, based on the total weight of the composition.

### Humectant

The compositions of the invention may comprise an additional humectant i.e. in addition to glycerol. Exemplary additional humectants useful in the present compositions include, but are not limited to, betaine, sarcosine, propylene glycol, butylene glycol, pentylene glycol, hexylene glycol, caprylyl glycol, sorbitol and glucose, and mixtures thereof. In one embodiment, the additional humectant is a mixture of pentylene glycol, caprylyl glycol and glucose.

Suitably, the additional humectant is present in an amount from about 1% to about 15% by weight, based on the total weight of the composition.

### Lip Conditioning Agent

The compositions of the invention may comprise a lip conditioning agent. Exemplary lip conditioning agents include, but are not limited to, capryloyl glycine, ceramide-3 and phytosphingosine, and mixtures thereof.

Suitably, the lip conditioning agent is present in an amount from about 0.001% to about 2% by weight, based on the total weight of the composition.

### pH Adjusting Agent

The compositions of the invention may further comprise a pH adjusting agent. In one embodiment, the pH adjusting agent is a base. Suitable bases include amines, bicarbonates, carbonates, and hydroxides such as alkali or alkaline earth metal hydroxides, as well as transition metal hydroxides. In an embodiment, the base is sodium hydroxide or potassium hydroxide. In one particular embodiment, the base is sodium hydroxide.

In another embodiment, the pH adjusting agent is an acid, an acid salt, or mixtures thereof. Suitably, the acid is selected from the group consisting of lactic acid, acetic acid, maleic acid, succinic acid, citric acid, benzoic acid, boric acid, sorbic acid, tartaric acid, edetic acid, phosphoric acid, nitric acid, ascorbic acid, dehydroacetic acid, malic acid, propionic acid, sulphuric acid and hydrochloric acid, or a combination or mixture thereof.

In yet another embodiment, the pH adjusting agent is a buffer. Suitably, the buffer is selected from the group consisting of citrate/citric acid, acetate/acetic acid, phosphate/phosphoric acid, propionate/propionic acid, lactate/lactic acid, carbonate/carbonic acid, ammonium/ammonia and edetate/edetic acid, or a combination or mixture thereof.

### Pharmaceutically Active Agent

The compositions of the invention may further comprise a pharmaceutically active agent. Exemplary pharmaceutically active agents include, but are not limited to, an anti-inflammatory agent, an antibacterial agent, an antiviral agent, a nutritional agent, an antioxidant, a sunscreen and a sun-blocking agent, and mixtures thereof. Suitably, the pharmaceutically active agent is present in an amount from about 0.001% to about 30% by weight, depending on the nature of the active agent, the condition being treated, and the composition.

In preferred embodiments, the present lip protectant compositions enhance the effectiveness of the pharmaceutically active agent. This enhanced effectiveness may result from an improved solubility profile of the pharmaceutically active agent.

In one embodiment, the pharmaceutically active agent is an anti-inflammatory agent. Exemplary anti-inflammatory agents are N-acylalkanolamines including, but not limited to, lactamide monoethanolamide (MEA), oleamide MEA, acetamide MEA (AMEA), palmitidyl MEA (PMEA), N-acetylphosphatidylethanolamine, N-acetylethanolamine, N-oleoylethanolamine, N-linolenoylethanolamine, N-acylethanolamine, and N-acyl-2-hydroxy-propylamine. In one embodiment, the N-acylalkanolamine is present in an amount from about 0.01% to about 2% by weight, based on the total weight of the composition.

In another embodiment, the pharmaceutically active agent is a sunscreen. Suitably, the sunscreen is a UVA sunscreen and/or a UVB sunscreen. Suitably, the sunscreen is a combination of a UVA sunscreen and a UVB sunscreen.

Human lips are prone to sun damage when exposed to UVA and/or UVB radiation. Efficacious protection from INA and UVB radiation requires the use of significant amounts of sunscreen, and often a mixture of organic sunscreens, to achieve efficacious protection from both UVA and UVB radiation. UVB radiation, which is radiation in the wavelength range of 290 nm to 320 nm, has traditionally been characterized as the radiation that causes sunburn. In addition, UVB radiation can decrease enzymatic and non-enzymatic antioxidants in the skin and impair the natural protective mechanisms in the skin, thereby contributing to DNA damage and potentially skin cancer. The dangers of UVA radiation, which is radiation in the wavelength range of 320 nm to 400 nm, have only recently been recognized. Chronic exposure to UVA radiation can cause damage to gene P53 DNA, possibly leading to cancer. Additionally, the longer UVA wavelengths allow for relatively deep penetration into the skin tissues causing damage to the elastic fibers and collagen which give skin its shape, thus causing wrinkling and eventually premature skin aging. Thus, protecting the lips from UVA and UVB radiation is important for skin health and overall health more generally.

Unfortunately, sunscreens, particularly organic sunscreens, have an unpleasant taste. Some sunscreens including Avobenzone which is particularly useful for UVA protection have a very unpleasant taste. This unpleasant taste is not an issue for lotions that are applied to the body to protect body surfaces from sun damage, but become a significant problem when sunscreens are incorporated into lip protectant compositions as described herein. Unfortunately, there are no other available sunscreens which afford UVA protection as effectively as Avobenzone.

Conventionally, sweeteners and/or flavorants have been used to cover or mask unpleasant tastes. In this approach, the sweetener and/or flavorant competes with the undesirable taste. While this may be successful in some applications, it is not satisfactory for masking the taste of the very strong and/or bitter flavors of organic sunscreens. Additionally, the flavor and/or sweetener may lack the persistence of taste over the entire time frame that the sunscreen remains on the lips, resulting in the evolution of a distasteful sensation after a period of time.

Coatings and forms of encapsulation are other approaches for taste-masking. However, coatings and/or encapsulation may impact the effectiveness of the sunscreen. Further, coating or encapsulation of an unpleasant tasting material in a lip protectant is typically an even more difficult problem than taste-masking of an ingested material, as unlike ingested materials, the product is intended to stay on the lips for a period of several hours.

Human skin is repeatedly exposed to ultraviolet radiation (UVR) that influences the function and survival of many cell types and is regarded as the main causative factor of skin cancer. It has been traditionally believed that skin pigmentation is the most important photoprotective factor, since melanin, besides functioning as a broadband UV absorbent, has antioxidant and radical scavenging properties. There are two types of melanin found in mammals that give hair and skin its distinctive coloring, the brownish black eumelanin and the reddish yellow pheomelanin. There are recent suggestions that pheomelanin, rather than protecting the skin against UV radiation, may actually contribute to UV-induced skin damage. (Thody et al., J. Invest Dermat 97:340-344 (1991)). Pheomelanin is also more concentrated on the lips in all individuals than eumelanin.

The shielding effect of melanin, especially eumelanin, is achieved by its ability to serve as a physical barrier that scatters UV radiation (UVR), and as an absorbent filter that reduces the penetration of UVR through the epidermis. The efficacy of melanin as a sunscreen was assumed to be about 1.5-2.0 sun protective factors (SPF); possibly as high as 4 SPF, implying that melanin absorbs 50% to 75% of UVR. In contrast to eumelanin, pheomelanin is especially prone to photodegradation and is thought to contribute to the damaging effects of UVR because it can generate hydrogen peroxide and superoxide anions and might cause mutations in melanocytes or other cells. See Brenner et al., Photochem Photobiol, 84(3): p 539-549 (2008).

A topical composition which not only moisturizes but protects the lips from UVA and UVB radiation and reduces pheomelanin damage will provide additional benefits to the patient. In particular, the present invention provides for a balanced UVA/SPF ratio of about 1:1 of sunscreen filters which is believed important to lip protection.

Another embodiment of the present invention is a method of protecting pheomelanin in the lips of a mammal from photodegradation, the method comprising applying to the lips of the mammal in need thereof an effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure; and
(e) at least one UVA sunscreen and at least one UVB sunscreen; and
   wherein the composition is a lip protectant composition.

Another embodiment of the disclosure is a method of protecting the lips of a mammal against reactivation of herpes simplex virus, the method comprising applying to the lips of the mammal in need thereof an effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure; and
(e) at least one UVA sunscreen and at least one UVB sunscreen; and wherein the UVA/SPF protection ratio is about 1:1; and
   wherein the composition is a lip protectant composition.

Yet another embodiment of the disclosure is a method of protecting the lips of a mammal against a reoccurrence of cold sores, the method comprising applying to the lips of the mammal in need thereof an effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;

(c) a thickening agent;
(d) at least one lamellar membrane structure; and
(e) at least one UVA sunscreen and at least one UVB sunscreen; and wherein the UVA/SPF protection ratio is about 1:1; and
   wherein the composition is a lip protectant composition.

UVA filters include, but are not limited to, Avobenzone (Parsol 1789), Bisdisulizole disodium (Neo Heliopan AP), Diethylamino hydroxybenzoyl hexyl benzoate (Uvinul A Plus), Ecamsule (Mexoryl SX), Menthyl anthranilate (Meradimate), oxybenzone, sulisobenzene and dioxybenzone, and mixtures thereof.

UVB filters include, but are not limited to, Amiloxate, 4-Aminobenzoic acid (PABA), Cinoxate, Ethylhexyl triazone (Uvinul T 150), Homosalate, 4-Methylbenzylidene camphor (Parsol 5000), Octyl methoxycinnamate (Octinoxate), Octyl salicylate (Octisalate), Padimate O (Escalol 507), Phenylbenzimidazole sulfonic acid (Ensulizole), Polysilicone-15 (Parsol SLX) and Trolamine salicylate, and mixtures thereof.

UVA+UVB filters include, but are not limited to, Bemotrizinol (Tinosorb S), Benzophenones 1-12, Dioxybenzone, Drometrizole trisiloxane (Mexoryl XL), Iscotrizinol (Uvasorb HEB), Octocrylene, Oxybenzone (Eusolex 4360), Sulisobenzone and Bisoctrizole (Tinosorb M), and mixtures thereof.

Other exemplary sunscreens useful in the present invention (with maximum suitable amounts of each sunscreen in % wt/wt) include, but are not limited to, amino benzoic acid (about 15%), Avobenzone (about 3%), cinoxate (about 3%), octyl methoxycinnamate (Octinoxate) (about 10%), homosalate (about 15%), meradimate (about 5%), octocrylene (about 10%), ethylhexyl salicylate (also known as octyl salicylate or octisalate) (about 5%), oxybenzone (about 6%), dioxybenzone (about 3%), Octyldimethyl PABA (Padimate O) (about 8%), p-amyldimethyl PABA (Padimate A) (about 3%), Phenylbenzimidazole sulfonic acid (ensulizole)(about 4%), sulisobenzene (about 10%), trolamine salicylate (about 12%), benzophenone (about 10%), benzylidine compounds, such as 4-methylbenzylidine camphor (Parsol 5000) (about 6%), butyl methoxydibenzoylmethane (about 5%), bis-ethylhexyloxyphenol methoxyphenyl triazone (Bemotrizinol or Tinosorb S) (about 10%), camphor benzalkonium methosulfate (about 6%), diethyl amino hydroxy benzoyl hexyl benzoate (Uvinul A plus) (about 10%), diethylhexyl butamido triazone (Uvasorb HEB) (about 10%), disodium phenyl dibenzylmidazole tetrasulfonate (Bisdisulizole disodium or NeoHeliopan AP) (about 10%), drometrizole trisiloxane (silatriazole or Mexoryl XL) (about 15%), ethylhexyl dimethyl para-amino benzoic acid (about 8%), ethylhexyl methoxycinnamate (about 10%), ethylhexyl Triazone (Uvinul T 150) (about 5%), isoamyl p-methoxycinnamate (about 10%), 4-methylbenzylidene camphor (about 10%), methylene bis-benzotriazolyl tetramethylbutylphenol (Bisoctrizole or Tinosorb M) (about 10%), PEG-25 paramainobenzoic acid (about 5%), phenylbenziamido methylbenzylidene camphor (about 6%), diisopropyl methyl cinnamate (about 10%), dimethoxyphenyl-[1-(3,4)-4,4-dimethyl]1,3 pentanedione (about 7%), ethylhexyl dimethyloxy benzylidene dioxoimidazoline propionate (about 3%), ferulic acid (about 10%), glyceryl ethylhexanoate dimethoxycinnamate (about 10%), glycerol para-aminobenzoic acid (about 10%), phenylbenzimidazole sulfonic acid (about 3%) and Parasol SLX (benzylidene malonate polysiloxane), and mixtures thereof. The amounts listed in the preceding list are for each sunscreen individually. In some embodiments in which a combination or mixture of sunscreens is used, the total combined amount of a sunscreen may be less or equal to the sum of the maximum suitable amounts for each individual sunscreen.

As used herein, the term “Cinnamates”, include octinoxate, cinoxate, and isoamyl p-methoxy cinnamate.

As used herein, the term “Salicylates” include octisalate, homosalate, and trolamine salicylate. As used herein, the term “Benzophenones” includes oxybenzone, sulisobenzone, and dioxybenzone.

As used herein, the term “PABA and derivatives” includes PABA (p-aminobenzoic acid), Octyldimethyl PABA (Padimate O), p-amyldimethyl PABA (Padimate A), Ethyl 4[bis(hydroxypropyl)]aminobenzoate, and glyceryl PABA.

Avobenzone, and benzophenones, as well as some other sunscreens, are photo unstable. Therefore these sunscreens are frequently combined with other sunscreens or stabilizers to increase the photostability of the final product. Some suitable photo stabilizers also referred to herein as boosters, include, but are not limited to Octocrylene, Diethylhexyl 2,6-naphthalate, and Diethylhexyl syringylidene malonate. In one embodiment, the photostabilizer is Diethylhexyl syringylidene malonate.

Although a single sunscreen may be used in a lip protectant composition, typically a combination of sunscreens will be used as each sunscreen has a characteristic wavelength range in which it absorbs UV radiation (UVR) and typically that range is less than the entire range for which protection is desired, Thus, use of a combination of sunscreens provides protection over a wider range of wavelengths. Additionally, efficacy of protection is also related to the amount of sunscreen. As regulatory agencies limit the amount of each sunscreen that can be used, the use of multiple sunscreens improves the SPF while maintaining regulatory compliance.

Organic sunscreens and their efficacious wavelength range (along with suitable amounts) are as follows: amino benzoic acid (260 nm-313 nm, about 5% to about 15%); padimate O (290 nm-315 nm, about 1.4% to about 8%); dioxybenzone (260 nm-380 nm, about 1% to about 3%); oxybenzone (270 nm-350 nm, about 2% to about 6%); sulisobenzone (260 nm-375 nm, about 5% to about 10%); cinoxate (270 nm-328 nm, about 1% to about 3%); octocrylene (250 nm-360 nm, about 7% to about 10%); avobenzone (320 nm-400 nm, about 1% to about 3%); octyl salicylate (280 nm-320 nm, about 3% to about 5%); homosalate (295 nm-315 nm, about 4% to about 15%); trolamine salicylate (260 nm-320 nm, about 5% to about 12%); octinoxate (290 nm-320 nm, about 2% to about 7.5%).

In one embodiment, at least two sunscreens are used where the first sunscreen has an efficacious wavelength range that includes about 280 urn to about 315 nm and the second sunscreen has an efficacious wavelength range that includes about 315 nm to about 400 nm. In one embodiment, the at least one UVA sunscreen is Avobenzone. In an embodiment, the at least one UVA sunscreen is Avobenzone and the composition further comprises a sunfilter stabilizer, suitably diethylhexyl syringylidene malonate. In another embodiment, the at least one UVB sunscreen is ethylhexyl salicylate (Octisalate). In yet another embodiment, the at least one UVB sunscreen is ethylhexyl salicylate (Octisalate) and the composition further comprises a sunfilter stabilizer, suitably diethylhexyl syringylidene malonate.

In one embodiment, the sunscreen is a combination of Avobenzone and ethylhexyl salicylate (Octisalate). In another embodiment, the sunscreen is a combination of Avobenzone and ethylhexyl salicylate (Octisalate), and the composition further comprises a sunfilter stabilizer, suitably diethylhexyl syringylidene malonate.

In one embodiment, the UVA/SPF protection ratio is about 1:1 to about 1:3. In another embodiment, the UVA/SPF protection ratio is about 1:1. To determine this number in vivo testing is performed for the SPF value and in vitro testing is performed for the UVA value. The UVA number is divided by the SPF number to obtain the protection value, for instance, a UVA value of 10 and a SPF of 10 would yield a 1/1 value. For instance, using a formulation described herein the ratio was found to be 10.8:12.1 or about 0.9:1. For purposes herein, this will be representative of a UVA/SPF protection ratio of about 1:1.

In one embodiment, the Avobenzone is present in an amount from about 2% to about 3% by weight, based on the total weight of the composition. In another embodiment, Octisalate is present in an amount from about 4% to about 5% by weight; based on the total weight of the composition. In yet another embodiment, Avobenzone is present in an amount from about 2% to about 3% by weight and Octisalate is present in an amount from about 4% to about 5% by weight, based on the total weight of the composition. In another embodiment, Avobenzone is present in an amount of about 2.8% and Octisalate is present in an amount of about 4.6%, based on the total weight of the composition.

The use of Avobenzone is particularly desirable for UVA protection as it is efficacious in the range of about 320 nm to 400 nm, a range in which most sunscreens provide limited to no protection. However, Avobenzone has particularly offensive organoleptic properties. The present invention provides not only for the use of efficacious amounts of Avobenzone in a lip protectant but in a composition which covers the offensive taste.

In some embodiments of the invention, it may be desirable to also include inorganic sunscreens such as titanium dioxide and/or zinc oxide, for example. Such compounds may be used in amounts from about 2% to about 25% by weight, with higher amounts providing higher levels of protection. Unfortunately, although higher amounts of inorganic oxides provide better protection, they typically also impart a thick layer of white material on the skin's surface which is very undesirable on the lips. Thus for lip protectant compositions, inorganic sunscreens are preferably used in combination with organic sunscreens to obtain efficacious protection.

Accordingly, the compositions of the invention have a comparatively high water content (relative to the prior art) and therefore enhance the moisturization of the lips. They are also generally free of conventional emulsifiers and thus are capable of restoring or repairing the skin lipid barrier of the lips. Furthermore, in some embodiments, the compositions protect the lips from UV damage. More specifically, the compositions are formulated to protect the lips from UVA radiation and thus assist in preventing photodegradation of pheomelanin in the lips. The U.S. Skin Protectant monograph requires a high level of glycerin, e.g. 20% to 45% to be compliant with the monograph and be considered a lip protectant. This is very difficult to achieve in a formulation which is not tacky and still consumer friendly to use. The monograph can be found at http://www.accessdata.fda.gov/scripts/cdrh/cfdocs/cfCFR/CFRSearch.cfm?CFRPart=347.

In one embodiment of the disclosure wherein the compositions comprise from about 20% to about 40% glycerin, they are in accordance with the monograph requirements. Accordingly, the present lip protectant compositions are believed to be highly advantageous, not only as a lip protectant but as a protectant against UV damage.

The present compositions are also capable of providing significant and perhaps extended moisturization.

In one embodiment, the invention provides a topical oil-in-water emulsion composition comprising:
(a) a discontinuous oil phase;
(b) a continuous aqueous phase comprising water and glycerin, wherein the glycerin is present in an amount from about 12% to about 40% by weight, based on the total weight of the composition;
(c) a thickening agent; and
(d) at least one lamellar membrane structure, comprising a phospholipid and water; and
   wherein the composition is a lip protectant composition.

In another embodiment, the invention provides a topical oil-in-water emulsion composition comprising:
(a) a discontinuous oil phase;
(b) a continuous aqueous phase comprising water and glycerin, wherein the glycerin is present in an amount from about 12% to about 40% by weight, based on the total weight of the composition;
(c) a thickening agent;
(d) at least one lamellar membrane structure, comprising a phospholipid, water, and a lipid;
(e) optionally a ceramide; and
(f) optionally at least one dermatologically acceptable excipient; and
   wherein the composition is a lip protectant composition.

In yet another embodiment, the invention provides a topical oil-in-water emulsion composition comprising:
(a) a discontinuous oil phase;
(b) a continuous aqueous phase comprising water and glycerin, wherein the glycerin is present in an amount from about 12% to about 40% by weight, based on the total weight of the composition;
(c) a thickening agent;
(d) at least one lamellar membrane structure, comprising a phospholipid, water, a lipid, and a phytosterol, and optionally at least one of squalane, rice bran oil, rice bran wax, pentylene glycol and/or hexylene glycol, and a ceramide; and
(e) optionally at least one dermatologically acceptable excipient; and
   wherein the composition is a lip protectant composition.

In a further embodiment, the invention provides a topical oil-in-water emulsion composition comprising:
(a) a discontinuous oil phase;
(b) a continuous aqueous phase comprising water and glycerin, wherein the glycerin is present in an amount from about 12% to about 40% by weight, based on the total weight of the composition;
(c) a thickening agent;
(d) at least one lamellar membrane structure, comprising a phospholipid, water, a phytosterol, squalane, rice bran oil and rice bran wax;
(e) optionally a ceramide; and
(f) at least one dermatologically acceptable excipient; and
   wherein the composition is a lip protectant composition.

In yet a further embodiment, the invention provides a topical oil-in-water emulsion composition comprising:
(a) a discontinuous oil phase;
(b) a continuous aqueous phase comprising water and glycerin, wherein the glycerin is present in an amount from about 12% to about 40% by weight, based on the total weight of the composition;
(c) a thickening agent;
(d) at least one lamellar membrane structure; and
(e) a combination of a UVA sunscreen and a UVB sunscreen, and wherein the UVA/SPF protection ratio is about 1:1; and
   wherein the composition is a lip protectant composition.

In another embodiment, the invention provides a topical oil-in-water emulsion composition comprising:
(a) a discontinuous oil phase;
(b) a continuous aqueous phase;
(c) a thickening agent;
(d) at least one lamellar membrane structure, comprising a phospholipid, water, and at least one of rice bran oil and rice bran wax; and
(e) optionally at least one dermatologically acceptable excipient.

Another embodiment of the disclosure is a novel lamellar membrane structure concentrate composition which comprises at least one lamellar membrane structure, comprising a phospholipid, water, and at least one of rice bran oil and rice bran wax; and optionally at least one of a lipid, squalane, a phytosterol, cholesterol or cholesterol derivative, a ceramide, or a triglyceride.

In one embodiment, the invention provides a topical oil-in-water emulsion composition comprising:

| | |
|---|---|
| Water | about 24.1% |
| Glycerin | about 21.3% |
| Glucose | about 11.0% |
| Diethylhexyl syringylidene malonate | about 10.0% |
| Butyrospermum Parkii (Shea) butter | about 7.8% |
| Olus (vegetable) oil | about 7.0% |
| Octisalate (UV Filter) | about 4.6% |
| Oryza Sativa (Rice) bran wax | about 3.1% |
| Avobenzone (UV Filter) | about 2.8% |
| Behenyl alcohol | about 2.0% |
| Caprylic/capric triglyceride | about 1.5% |
| Oryza Sativa (Rice) bran oil | about 1.1% |
| Capryloyl glycine | about 1.0% |
| Hydrogenated lecithin | about 0.9% |
| Pentylene glycol | about 0.8% |
| Flavor | about 0.1% |
| Caprylyl glycol | about 0.2% |
| Dehydroxanthan gum | about 0.2% |
| Sodium hydroxide | about 0.2% |
| Squalane | about 0.2% |
| Tocopherol | about 0.1% |
| VP/Eicosene copolymer | about 0.1% |
| Sodium carbomer | about 0.1% |
| Acrylates/C10-30 Alkyl Acrylate crosspolymer | about 0.1% |
| Trisodium ethylenediamine disuccinate | about 0.02% |
| Palmitamide MEA | about 0.02% |
| Ascorbyl palmitate | about 0.01% |
| Ceramide-3 | about 0.002% |
| Phytosphingosine | about 0.002% |

and wherein all percentages are based on the percent by weight of the final composition, and all totals equal 100% by weight, and wherein the composition is a lip protectant composition.

### Methods of Treatment

The invention provides a method for moisturizing, and protecting, repairing, or restoring the skin lipid barrier of the lips of a mammal, the method comprising applying to the lips of the mammal in need thereof an effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent; and
(d) at least one lamellar membrane structure; and
   wherein the composition is a lip protectant composition.

The invention also provides for the use of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent; and
(d) at least one lamellar membrane structure;
   in the manufacture of a lip protectant composition for the moisturizing, and protecting, repairing, or restoring the skin lipid barrier of the lips of a mammal.

The invention further provides for the use of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent; and
(d) at least one lamellar membrane structure; and
   wherein the composition is a lip protectant composition, for moisturizing, and protecting, repairing, or restoring the skin lipid barrier of the lips of a mammal.

The invention further provides for the use of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure; and
(e) at least one UVA sunscreen and at least one UVB sunscreen; and wherein the UVA/SPF protection ratio is about 1:1; and
   wherein the composition is a lip protectant composition, for protecting the lips of a mammal with broad spectrum protection of a UVA sunscreen and a UVB sunscreen, and enriched in UVA protection.

The protection and repair of the skin lipid barrier by the compositions of the present invention improves the skin barrier function and conveys numerous additional therapeutic effects to a mammal to which the compositions are applied.

In one embodiment of the disclosure, the compositions described herein provide moisturization to the lips. It has unexpectantly been found that the substantial amount of glycerin present in the aqueous phase does not make the composition feel tacky or sticky in comparison to other compositions with less glycerin present.

The compositions of the invention are applied to the lips at a frequency consistent with the condition of the lips. For example, where the lips are irritated and in need of repair, more frequent application may be required. Alternatively, where the lips are not irritated and the composition is being applied to merely protect the barrier function of the lips, less frequent application may be possible.

### DEFINITIONS

The term “applying” as used herein refers to any method which, in sound medical or cosmetic practice, delivers the topical composition to the lips of a subject in such a manner so as to provide a positive effect on a dermatological disorder, condition, or appearance. The compositions are preferably administered such that they cover the entire lips.

As used herein, the phrases an “effective amount” or a “therapeutically effective amount” refers to an amount of a composition or component thereof sufficient enough to have a positive effect on the area of application. Accordingly, these amounts are sufficient to modify the skin disorder, condition, or appearance to be treated but low enough to avoid serious side effects, within the scope of sound medical advice. An effective amount will cause a substantial relief of symptoms when applied repeatedly over time. Effective amounts will vary with the particular condition or conditions being treated, the severity of the condition, the duration of the treatment, and the specific components of the composition being used.

An “effective amount” of a sunscreen is an amount of sunscreen sufficient to provide measurable protection from solar radiation as determined by having a measurable Sun Protection Factor (SPF) value and/or UVA protection value.

The term “SPF” (Sun Protection Factor) means the UVB energy required to produce a minimal erythema dose on sunscreen treated skin divided by the UVB energy required to produce a minimal erythema dose on unprotected skin.

As used herein, “treatment” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

As indicated above, “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

The phrase “dermatologically acceptable excipient” as used herein refers to any inactive ingredient present in the herein described compositions. Each excipient must be compatible with the other ingredients of the lip protectant composition when commingled such that interactions which would substantially reduce the efficacy of the composition when administered to an individual and interactions which would result in compositions that are not pharmaceutically or cosmetically acceptable are avoided. In addition, each excipient must be of sufficiently high purity to render it pharmaceutically or cosmetically acceptable.

As used herein, a “lip protectant” is a semisolid composition for application to the lips that provides protective, restorative and/or moisturizing properties. These compositions include creams, and lip balms in a stick presentation, as well as soft lip balms such as, for example, those dispensed from jars, pots or tubes.

The term “stick lip balm” means a lip balm that can be formed into a stick that is extensible and retractable from a container and is sufficiently robust to substantially retain the stick shape under typical commercial conditions of shipping, storage and use.

Lip balms are an over-the-counter drug defined as a “drug product that relieves and prevents dryness or chapping of the exposed surface of the lip”. Fed Reg. Skin Protectant Drug Products, Final Rules, Jun. 4, 2003 Vol. 68, No. 107, pp 3362-3381.

The term “lipstick” means a waxy stick product containing pigment which is transferable to the lips to impart a visible color. Lipsticks may be cosmetics or lip treatments. A lipstick is a lip treatment if, in addition to imparting color, it provides protective and/or moisturizing properties, and/or a beneficial agent and/or a sunscreen and/or a pharmaceutically active agent to the lips or lip area.

The term “organic sunscreen” means a compound or mixture of compounds that can protect human skin from UVA and/or UVB radiation and is the class of compounds classified by those skilled in the art of chemistry as organic chemicals.

The term “inorganic sunscreen” means a compound or mixture of compounds that can protect human skin from UVA and/or UVB radiation and is the class of compounds classified by those skilled in the art of chemistry as inorganic chemicals. Exemplary inorganic sunscreens include, but are not limited to, zinc oxide and titanium dioxide.

The term “about” means within an acceptable range for the particular parameter specified as determined by one of ordinary skill in the art, which will depend, in part, on how the value is measured or determined, i.e., the limitations of the measurement system. For example, “about” can mean a range of up to 10% of a given value.

“%” as used herein, refers to the percentage by weight of the total composition, unless otherwise specified. All percentages are based on the percent by weight of the final composition prepared unless otherwise indicated and all totals equal 100% by weight.

The term “wt/wt” or “by weight”, unless otherwise indicated, means the weight of a given component or specified combination of components to the total weight of the composition expressed as a percentage.

A designation that a substance is a semisolid, should be taken to mean the physical state of the substance in the temperature range of about 20° C. to about 40° C.

As used herein, the term “phytosterol” refers to plant sterols and plant stanols. Plant sterols are naturally occurring cholesterol-like molecules found in all plants, with the highest concentrations occurring in vegetable oils. Plant stanols are hydrogenation compounds of the respective plant sterols. Phytosterols are natural components of common vegetable oils.

As used herein, the term “sensitive skin” refers to the degree of skin irritation or skin inflammation, as exemplified by parameters in suitable assays for measuring sensitivity, inflammation or irritation.

It should be understood that the terms “a” and “an” as used herein refer to “one or more” of the enumerated components. It will be clear to one of ordinary skill in the art that the use of the singular includes the plural unless specifically stated otherwise.

The term “and/or” as used herein covers both additively and also alternatively the individual elements of a list which are thus linked so that these elements are to be understood as linked selectively with “and” or respectively with “or”. Furthermore, the terms used in the singular of course also comprise the plural.

Throughout the application, descriptions of various embodiments use “comprising” language, however in some specific instances, an embodiment can alternatively be described using the language “consisting essentially of” or “consisting of”.

“Substantially free” of a specified component refers to a composition with less than about 1% by weight of the specified component. “Free” of a specified component refers to a composition where the specified component is absent.

As used herein, “mammal” includes but is not limited to humans, including pediatric, adult and geriatric patients.

The following examples are illustrative of the present invention and are not intended to be limitations thereon.

Other terms as used herein are meant to be defined by their well-known meanings in the art.

### EXAMPLES

### Example 1A

### Lip Protectant Composition

A lip protectant composition having the following formulation was prepared, Table 3:

| Ingredients | % w/w |
|---|---|
| Lamellar membrane structure concentrate* | 15.000 |
| DL-Alpha tocopherol | 0.100 |
| Glycerol | 20.670 |
| Ascorbyl palmitate | 0.010 |
| Butyrospermum Parkii | 7.500 |
| Behenyl alcohol | 2.000 |
| Oryza Sativa Cera | 3.000 |
| VP/Eicosene copolymer | 0.100 |
| Diethylhexyl Syringylidene Malonate | 11.100 |
| (9.990%) | |
| Caprylic/Capric triglyceride (1.110%) | |
| Butyl methoxydibenzoylmethane | 2.780 |
| (Avobenzone) | |
| Ethylhexyl salicylate (Octisalate) | 4.550 |
| Olus (vegetable) oil | 7.000 |
| Sodium carbomer | 0.050 |
| Acrylates/C10-30 alkyl acrylate | 0.050 |
| crosspolymer | |
| Trisodium ethylenediamine disuccinate | 0.050 |
| Sodium hydroxide pellets | 0.185 |
| Glucose monohydrate | 12.000 |
| Capryloyl glycine | 1.000 |
| Caprylyl glycol | 0.200 |
| Dehydroxanthan gum | 0.200 |
| Cool mint flavour | 0.500 |
| Water (purified) | 11.955 |
| Total | 100.000 |

The composition was prepared in two key steps. In the first step, a concentrate having the lamellar membrane structure* composition was prepared (see Table 4). The concentrate comprises hydrogenated lecithin, palmitamide MEA, Oryza Sativa bran oil, Oryza Sativa Cera, Butyrospermum parkii butter, squalane, pentylene glycol, glycerin, phytosphingosine, ceramide 3 and water. In a second step, the concentrate is added during the formulation of an oil-in-water emulsion to give the final composition as follows:

### Phase 1 (Aqueous):

11.955% by weight Water

0.200% by weight Caprylyl glycol

12.000% by weight Glucose monohydrate

0.200% by weight Dehydroxanthan gum

20.670% by weight Glycerin

0.185% by weight NaOH

1.000% by weight Capryloyl glycine

### Phase 2 (Oil):

7.500% by weight Butyrospermum parkii butter

2.000% by weight Behenyl alcohol

3.000% by weight Oryza sativa cera

0.100% by weight VP/eicosene copolymer

11.100% by weight Diethylhexyl syringylidene malonate/caprylic/capric triglyceride

2.780% by weight Butyl methoxydibenzoylmethane

0.010% by weight Ascorbyl palmitate

4.550% by weight Ethylhexyl salicylate

### Phase 3 (Thickening):

7.000% by weight Olus (vegetable) oil

0.050% by weight Sodium carbomer

0.050% by weight Acrylates/C10-30 alkyl acrylate crosspolymer

### Phase 4 (Lamellar Membrane Structure Component):

0.100% by weight Tocopherol

0.050% by weight Trisodium ethylenediamine disuccinate

0.500% by weight Flavor

15.000% by weight lamellar membrane structure concentrate

Phase 1 and Phase 2 were first heated to 80° C. for production. Phase 2 was then slowly added to Phase 1 while the temperature was maintained and the mixture was continuously stirred. The combined Phases were then homogenized for a minimum of 10 minutes at 3000 RPM in a Becomix. The combined phases were then cooled to 70° C. with continuous stirring. Phase 3 was then added to the combined Phases 1 and 2 and mixed for a minimum of 5 minutes. The combined Phases (1, 2, and 3) were then cooled to 35° C. with continuous stirring. Phase 4 was then added to Phases 1, 2, and 3 with continuous stirring. The mixture was then homogenized again for a minimum of 20 minutes at 3000 RPM. The lip protectant composition which was thus produced was able to be used directly.

The lamellar membrane structure concentrate*of Formula 1A has the following formulation:

TABLE 4

| Ingredients | % w/w |
|---|---|
| Water | 10.96578 |
| Oryza Sativa (Rice) bran oil | 1.05000 |
| Hydrogenated lecithin | 0.90000 |
| Pentylene glycol | 0.75000 |
| Glycerin | 0.74625 |
| Butyrospermum Parkii (Shea) butter | 0.30000 |
| Squalane | 0.15000 |
| Oryza Sativa (Rice) bran wax | 0.12000 |
| Palmitamide MEA | 0.01500 |
| Ceramide-3 | 0.00150 |
| Phytosphingosine | 0.00147 |
| Total | 15.00000 |

The final lip protectant composition has the following formulation:

TABLE 5

| Ingredients | % w/w |
|---|---|
| Water | 24.086 |
| Glycerin | 21.313 |
| Glucose | 10.980 |
| Diethylhexyl syringylidene malonate | 9.990 |
| Butyrospermum Parkii (Shea) butter | 7.800 |
| Olus (vegetable) oil | 7.000 |
| Octisalate (UV Filter) | 4.550 |
| Oryza Sativa (Rice) bran wax | 3.120 |
| Avobenzone (UV Filter) | 2.780 |
| Behenyl alcohol | 2.000 |
| Caprylic/capric triglyceride | 1.535 |
| Oryza Sativa (Rice) bran oil | 1.050 |
| Capryloyl glycine | 1.000 |
| Hydrogenated lecithin | 0.900 |
| Pentylene glycol | 0.750 |
| Flavor | 0.075 |
| Caprylyl glycol | 0.200 |
| Dehydroxanthan gum | 0.190 |
| Sodium hydroxide | 0.185 |
| Squalane | 0.150 |
| Tocopherol | 0.100 |
| VP/Eicosene copolymer | 0.100 |
| Sodium carbomer | 0.050 |
| Acrylates/C10-30 Alkyl Acrylate | 0.050 |
| crosspolymer | |
| Trisodium ethylenediamine disuccinate | 0.0185 |
| Palmitamide MEA | 0.0150 |
| Ascorbyl palmitate | 0.0099 |
| Ceramide-3 | 0.0015 |
| Phytosphingosine | 0.00147 |
| Total | 100.000 |

### Examples 1B-1J

### Lip Protectant Compositions

The following additional formulations (Examples 1B-1 J) were prepared using a similar approach:

TABLE 6
Examples 1B-1E

| | Example | | | |
|---|---|---|---|---|
| | 1B | 1C | 1D | 1E |
| | % | % | % | % |
| Ingredients | w/w | w/w | w/w | w/w |
| Lamellar membrane | 15.000 | 15.000 | 15.000 | 15.000 |
| structure concentrate | | | | |
| DL-Alpha tocopherol | 0.100 | 0.100 | 0.100 | 0.100 |
| Glycerol | 20.670 | 20.000 | 20.670 | 20.670 |
| Ascorbyl palmitate | 0.010 | 0.010 | 0.010 | 0.010 |
| Butyrospermum parkii | 7.500 | 7.500 | 7.500 | 7.500 |
| Behenyl alcohol | 2.000 | 2.000 | 2.000 | 2.000 |
| Oryza Sativa Cera | 3.000 | 3.000 | 3.000 | 3.000 |
| VP/Eicosene copolymer | 0.100 | 0.100 | 0.100 | 0.100 |
| Diethylhexyl | 11.100 | 11.100 | 11.100 | 11.100 |
| Syringylidene | | | | |
| Malonate (9.990%) | | | | |
| Caprylic/Capric | | | | |
| triglyceride (1.110%) | | | | |
| Butyl Methoxy- | 2.780 | 3.000 | 2.750 | 2.750 |
| dibenzoylmethane | | | | |
| (Avobenzone) | | | | |
| Ethylhexyl salicylate | 4.550 | 5.000 | 2.000 | 4.250 |
| (Octisalate) | | | | |
| Octocrylene | — | — | 2.500 | 3.000 |
| Benzophenone-3 | — | — | 2.500 | — |
| Olus (vegetable) oil | 7.000 | 7.000 | 4.580 | 4.330 |
| Sodium carbomer | 0.050 | 0.050 | 0.050 | 0.050 |
| Acrylates/C10-30 | 0.050 | 0.050 | 0.050 | 0.050 |
| alkyl acrylate | | | | |
| crosspolymer | | | | |
| Trisodium | 0.050 | 0.050 | 0.050 | 0.050 |
| ethylenediamine | | | | |
| disuccinate | | | | |
| Sodium hydroxide | 0.190 | 0.185 | 0.185 | 0.185 |
| pellets | | | | |
| Glucose monohydrate | 12.000 | 12.000 | 12.000 | 12.000 |
| Capryloyl glycine | 1.000 | 1.000 | 1.000 | 1.000 |
| Caprylyl glycol | 0.200 | 0.200 | 0.200 | 0.200 |
| Dehydroxanthan gum | 0.200 | 0.200 | 0.200 | 0.200 |
| Cool mint flavour | 0.500 | 0.500 | 0.500 | 0.500 |
| Water (purified) | 11.950 | 11.955 | 11.955 | 11.955 |
| Total | 100.000 | 100.000 | 100.000 | 100.000 |

TABLE 7
Examples 1F-1J

| | Example | | | | |
|---|---|---|---|---|---|
| | 1F | 1G | 1H | 1I | 1J |
| | % | % | % | % | % |
| Ingredients | w/w | w/w | w/w | w/w | w/w |
| Lamellar membrane | 15.000 | 15.000 | 15.000 | 15.000 | 15.000 |
| structure conc. | | | | | |
| DL-Alpha tocopherol | 0.100 | 0.100 | 0.100 | 0.100 | 0.100 |
| Glycerol | 20.000 | 20.000 | 20.000 | 20.000 | 20.670 |
| Ascorbyl palmitate | 0.010 | 0.010 | 0.010 | 0.010 | 0.010 |
| Butyrospermum parkii | 7.500 | 7.500 | 7.500 | 7.500 | 7.500 |
| Behenyl alcohol | 2.000 | 2.000 | 2.000 | 2.000 | 2.000 |
| Oryza Sativa Cera | 3.000 | 3.000 | 3.000 | 3.000 | 3.000 |
| VP/Eicosene copolymer | 0.100 | 0.100 | 0.100 | 0.100 | 0.100 |
| Diethylhexyl | — | — | — | — | 11.100 |
| Syringylidene | | | | | |
| Malonate (9.990%) | | | | | |
| Caprylic/Capric | | | | | |
| triglyceride (1.110%) | | | | | |
| Butyl Methoxy- | 2.500 | 2.500 | 2.500 | 2.500 | 2.780 |
| dibenzoylmethane | | | | | |
| (Avobenzone) | | | | | |
| Diethyl syringylidene | 2.000 | 8.000 | 8.000 | 8.000 | — |
| malonate | | | | | |
| Ethylhexyl | 2.500 | 2.500 | — | 5.000 | — |
| methoxycinnamate | | | | | |
| Octocrylene | 2.000 | 2.000 | 2.000 | 2.000 | — |
| Benzophenone-3 | 4.500 | 2.250 | 4.500 | — | — |
| Ethylhexyl salicylate | — | 2.000 | — | — | 4.550 |
| (Octisalate) | | | | | |
| Olus (vegetable) oil | 12.600 | 6.850 | 9.100 | 8.600 | 7.000 |
| Sodium carbomer | 0.050 | 0.050 | 0.050 | 0.050 | 0.050 |
| Acrylates/C10-30 | 0.050 | 0.050 | 0.050 | 0.05 | 0.050 |
| alkyl acrylate | | | | | |
| crosspolymer | | | | | |
| Trisodium | 0.050 | 0.050 | 0.050 | 0.050 | 0.050 |
| ethylenediamine | | | | | |
| disuccinate | | | | | |
| Sodium hydroxide | 1.850 | 1.850 | 1.850 | 1.85 | 0.185 |
| pellets | | | | | |
| Glucose monohydrate | 12.000 | 12.000 | 12.000 | 12.000 | 12.000 |
| Capryloyl glycine | 1.000 | 1.000 | 1.000 | 1.000 | 1.000 |
| Caprylyl glycol | 0.200 | 0.200 | 0.200 | 0.200 | 0.200 |
| Dehydroxanthan gum | 0.200 | 0.200 | 0.200 | 0.200 | 0.200 |
| Water (purified) | 10.790 | 10.790 | 10.790 | 10.790 | 12.455 |
| Total | 100.000 | 100.000 | 100.000 | 100.00 | 100.000 |

### Example 2

### Determining UVA Protection Factor and Critical Wavelength Value

The COLIPA method for in-vitro determination of UVA protection (March 2011) was used to determine the UVA protection factor and critical wavelength values of example formulations. This method is a laboratory method which requires an artificial ultraviolet (UV) light source with defined, known output and a Labsphere sunscreen analyzer to measure the absorbance spectra before and after UV irradiation.

The amount of test product corresponding to 1.3 mg/cm² was applied to 4 PMMA plates (HD-6, Helioscreen, Creil, France). The test product was applied by “spotting” the product on each plate and rubbing with a finger tip saturated with the test product for approximately one minute, then allowed to equilibrate in the dark for at least 30 minutes at 25° C.±2° C. A solar simulator (Model LS 10000-4S-0009, Solar Light Company, Philadelphia) that complied with Colipa specifications was used to irradiate the plates with a series of 4 UV doses (32, 64, 95, and 127 J/cm²) and a calibrated UV-2000 Sunscreen Analyzer (Model UV-2000S, Labsphere, North Sutton, N.H.) that complied with Colipa specifications was used to measure the sunscreen absorbance spectra on each plate, before UV irradiation and after each UV dose.

For each PMMA plate, the absorbance spectrum after a UV dose corresponding to 1.2×UVAPF₀ was computed by linear interpolation and used to obtain the UVAPF and critical wavelength. The critical wavelength is the wavelength at which the area under the absorbance spectrum reaches 90% of the total area under the absorbance spectrum. A critical wavelength of 370 nm or greater is required for labeling as providing “broad spectrum” protection.

TABLE 8

| | Mean | Mean | Mean Critical |
|---|---|---|---|
| Formulation | UVAPF₀ | UVAPF | Wavelength |
| Example 1A | 31.3 | 19.3 | 377 |
| Example 1B | 33.9 | 20.1 | 377 |
| Example 1C | 31.0 | 19.8 | 377 |
| Example 1D | 24.3 | 18.2 | 378 |
| Example 1E | 26.4 | 18.5 | 378 |
| Example 1F | 17.5 | 13.2 | 375 |
| Example 1G | 21.9 | 15.1 | 376 |
| Example 1H | 28.3 | 22.6 | 376 |

### Results:

These results indicate that the formulations will meet broad spectrum label requirements for the US (critical wavelength >370 nm). Additionally, the example formulations will more than meet the Colipa 1:3 UVA/UVB sun protection requirements.

### Example 3

### Determining the Sun Protection Factor (SPF)

The sun protection factor (SPF) was determined in vivo on the back of human subjects, according to the FDA Final Rule (2011) using a sun simulator. This method is an in vivo study which requires a sun simulator to supply an artificial ultraviolet (UV) light source with defined, known output. In conducting the study, a graduated series of delayed UV erythema reactions is induced on several small areas of skin on the back of selected subjects.

The subjects must present themselves at the study site at least three times:

Visit One: Subject's suitability is evaluated and their skin phototype is determined by colorimetric measurement. In order to establish the innate reactivity of each subject to UV radiation, a series of UV irradiations is carried out 24 hours before the actual examination (during the first visit). Each irradiation field is 1 cm in diameter. The time intervals are selected as a geometric series, wherein the irradiation duration is increased by 1.25× with each field. The irradiated areas are assessed 20±4 hours after UV exposure and the MEDu (MED of the unprotected skin) is determined. The MED (minimum erythema dose) serves as an indicator for the dose to be applied for the sun protection factor examination (SPF examination). The MED is defined as the irradiation energy which is required in order to produce a weak, but clearly discernible reddening of the skin with sharp delimitation. The irradiation dose in this examination was detected chronologically.

Visit Two: The negative control (untreated area) is irradiated, to detect the minimal erythemal dose of the unprotected skin (MEDu; u stands for “unprotected”)). The test materials are then applied to the test areas and a waiting time between 15 to 30 minutes is kept before starting irradiation of the test area with the sun simulator at an increment of 1.2×. By the gradual increase of the UV dose, different degrees of skin erythema are produced, which reach a maximum value approximately 24 hours after the UV exposure. Irradiation time is dependent on the expected SPF of the test materials, the skin phototype of the subject as detected by colorimetric measurements, determined MED after irradiation and the actual power of the sun simulator.

Visit Three: 20±4 hours after irradiation during visit two. The test areas are examined for each treatment to determine the protected Minimal Erythemal Dose (MEDp; p stands for “protected”). The MEDp is defined as the lowest UV dose that produces the first perceptible unambiguous erythema with defined boarders appearing over most of the field of UV exposure. The sun protection factor will be calculated by dividing the MEDp of the product treated test field by the MEDu of the untreated test field.

The MEDu and the MEDp can be evaluated visually by trained evaluators or instrumentally with a colorimeter. Several preparations can be tested here simultaneously on the same subject. A minimum of 10 valid results is sufficient. At most, three individual results may be excluded for documented reasons.

An examination of the test area is carried out on the subjects from the lower line of the shoulder blades down to waist height. Evidence of sunburn, suntan, scars, skin lesions, tattoos, scars, irritated skin, hairs, and irregular pigmentation is determined on the back of each subject. If, in the opinion of the examiner, one of the listed artifacts is present in a significant manner, the subject is excluded from the study. The examination was carried out on 13 subjects with Fitzpatrick skin phototypes I-III, see Fitzpatrick Table below.

TABLE 9
Fitzpatrick Classification Scale

| Skin Type | Skin Color | Characteristics |
|---|---|---|
| I | White; very fair; red or blond | Always burns, never tans |
| | hair; blue eyes; freckles | |
| II | White; fair; red or blond hair; | Usually burns, tans with |
| | blue, hazel or green eyes | difficulty |
| III | Cream white; fair with any eye | Sometimes mild burn, |
| | or hair color; very common | gradually tans |
| IV | Brown; typical Mediterranean | Rarely burns, tans with |
| | Caucasian skin | ease |
| V | Dark Brown; mid-eastern skin | Very rarely burns, tans |
| | types | very easily |
| VI | Black | Never burns, tans very |
| | | easily |

The UV source for the SPF study is a 300 W Multiport, SOLAR Light. The simulator is equipped with 6 irradiation fields which can emit different irradiation doses simultaneously.

The SPF for the compositions was determined at distinct positions on the backs of the subjects (n=13). Individual test areas (40 cm²) were outlined with a waterproof marker on the back of the subjects. The distance between different application sites of the test products was at least 1 cm to prevent test products from spreading over and influencing neighboring test sites.

The respective compositions were applied with a micro liter syringe to the test areas. The application dose is targeted as a quantity of 2 mg/cm²±0.05 mg/cm². After applying the test product to the test area, it will be quickly spread by gently rubbing (soft touch with light pressure) with a non-saturated finger-cot. Spreading time will be between 20 and 50 seconds. Following application, a waiting time between 15 to 30 minutes will be kept before starting irradiation of the test area with the sun simulator.

After the waiting time has elapsed, an unprotected area on the subject's back is irradiated (MEDu). Then the test is repeated on the areas treated with the respective composition (MEDp).

The test fields are treated with a series of UV irradiation units of different intensity. The actual exposure time is selected by means of the previously determined MED of the test person and of the assumed LPF of the product. More precisely, the MED is multiplied by the assumed SPF of the product; the exposure time results from this. For an expected SPF of 8 to 15 an increment of 1.2× will be chosen. The expected MED dose will be irradiated on the fourth step of the six irradiation doses. After completion of the irradiation, the position of the test fields is marked. Each subject is requested to cover the entire test area, to protect against further UV irradiation.

The evaluation of the treated and irradiated test fields was carried out by trained personnel 20±4 hours after UV exposure. The range of individual SPF values and mean SPF for the compositions are shown in the following table:

TABLE 10

| | Mean | Labeled |
|---|---|---|
| Formulation | SPF | SPF |
| Example 1A | 12.1 | SPF 10 |
| Example 1B | 13.8 | SPF 10 |
| Example 1D | 14.4 | SPF 12 |
| Example 1E | 15.5 | SPF 11 |

### Results:

These results indicate that the above example formulations have a labeled SPF within the range of SPF 10-SPF 12. This means that the example formulations will absorb 90% of UVB light.

### Overall Results (UVA+UVB Combined)

The formulations were customized for the lips to provide enriched UVA protection. All formulations detailed above provide at least a 1:1 UVA/SPF sun protection ratio. Typical sun filters provide more UVB protection than UVA protection. Use of the SPF value to describe sunscreen effectiveness is misleading. The SPF is primarily affected by UVB radiation and is not a significant indicator of protection against UVA radiation. There is a growing demand for a method of measuring the level of protection against UVA radiation (UVA Protection factor or UVA-PF). Recent guidance provided in the European Commission Recommendation of 22 Sep. 2006 on the efficacy of sunscreen products and the claims made relating thereto and harmonized against test methods specified in Colipa 2006/647/EC (see also Colipa 2011 and FDA Final Rule 2011) has created a standard where all marketed sunscreens must provide a UVA-PF which should equal at least ⅓ the sun protection factor as determined by the in vivo PPD (persistent pigment darkening) method or an equivalent degree of protection obtained by any in vitro method. The formulations detailed in the examples surpass this requirement by providing a balanced UVA/UVB protection. Most sunscreens (especially those formulated for the lips) only provide high levels of UVB protection, which leaves the skin and lips vulnerable to UVA damage. Thus, the present invention provides for a UVA/SPF ratio of at least 1:3 and preferably a UVA/SPF ratio of about 1:1.

### Importance of Enriched UVA Protection for the Lips

Melanocyte cells make pigment by creating one of two types of melanin: eumelanin or pheomelanin. Both are found in the skin including the lips and each creates different shades of pigment. Eumelanin is brown to black in color and is more common in those with a tan or darker skin. It also absorbs UVA rays (melanin absorbs at 335 nm, UVA spectrum), acting as a skin protector. Pheomelanin is yellow to red in color and is found in concentrated amounts in the lips and in people with lighter skin tones. Pheomelanin cannot absorb UVA rays and makes the skin more sensitive to UVA rays. Because of the enriched levels of pheomelanin found in the lips, lips are additionally vulnerable to UVA damage.

Additionally, pheomelanin is not able to neutralize ROS and is photodamaging in the presence of UVA radiation.

### Example 4

### Determining the Emulsion Ultra-Structure of Example 1B

The ultrastructure of Example 1B was investigated using cryo-TEM (transmission electron microscopy). Cryo-TEM is a technique that visualizes frozen-hydrated specimens. Imaging frozen-hydrated specimen enables the visualization of the skin-similar lamellar ultra-structure which is enabled/created by the inclusion of the Probiol concentrate.

To obtain an accurate image from cryo-TEM, the water in frozen-hydrated specimens must be in “vitreous” (glass-like) form. Ice crystals would disrupt the cryo-TEM image. To enable the water to freeze rapidly enough to produce the vitreous state, a thin layer of the emulsion must be plunged into a suitable cryogen. The maximum specimen thickness for the plunging technique is about 1 μm. The cryogen of choice is liquid ethane, cooled by liquid nitrogen. The desired water layer thickness is achieved by blotting the grid after application of a drop of solution. Filter paper type, blotting time, and humidity of the air surrounding the grid, all affect the thickness of the frozen water layer. Many techniques have been designed to maintain specimen integrity during preparation, transfer and observation. For the vitrification process as such, the main parameters to control are humidity and temperature.

### Results:

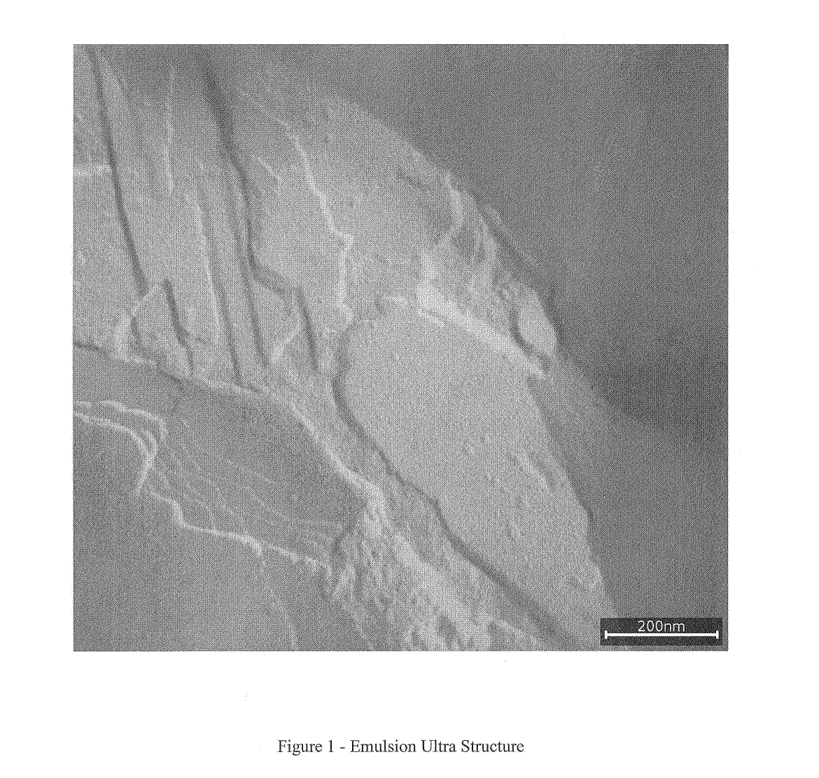

The cryoTEM images clearly demonstrate the presence of lamellar sheets in the emulsion structure. These lamellar sheets are similar in structure to the lipid layer of the stratum corneum. See FIG. 1.

### Example 5

### Visualization of Lipid and UV Filter Components

Coherent Antistokes Raman Scattering (CARS) is a form of spectroscopy that is sensitive to the vibrational signatures of molecules, typically the nuclear vibrations of chemical bonds. CARS has been used extensively for non-invasive imaging of lipids in biological samples. Here we describe the use of CARS to develop a 3D chemical map that was tuned to show areas of lipid and areas corresponding to the UV filter components of the compositions.

All Coherent Antistokes Raman Scattering (CARS) experiments were carried out on a Leica TCS-SP8 microscope with an AOBS detection system. Samples were prepared by placing approximately 10 μL of product on a cleaned microscope slide which was then covered with a coverslip. Slight pressure was applied to the coverslip to produce a thin film of product between the microscope slide and coverslip. The sample was then mounted in the microscope, and focused using a 40× objective lens. For CARS measurement of the lipid bands, the laser was tuned to enable detection of the 2850 cm⁻¹ vibrational band which corresponds to the presence of CH₂ groups. For analysis of the UV filter components in the product, the laser was re-tuned to excite the 1590 cm⁻¹ band, which is a second harmonic of benzene rings (present within the UV filters). A beam splitter inside the microscope was then activated to enable this to pass through the objective lens and into the sample. Backscattered light then passed back through the objective lens and was collected to enable generation of the CARS map of the sample. Rastering of the light across the sample enables a 2D distribution of lipid and/or UV filters to be determined, and movement of the position of the sample in relation to the objective lens allowed the 3D distribution of components within the sample to be determined. Leica image analysis software was then used to compile the data from each individual slice into a 3D map. In the 3D map, areas which have lipid present are colored in green, and areas which are devoid of lipid (i.e. have water present) are colored in black. UV filters (where analyzed) are colored in red.

TABLE 11
Formulations Tested

Example 1A
Example 1B
Example 1F
Example 1G
Example 1H
Example 1I
Example 1J

All formulations tested showed the typical structure of oil-in-water emulsions (droplets of oily, lipid material present in a water matrix). The formulations all showed strong bands at 1590 cm⁻¹ (UV filter, red) and 2850 cm⁻¹ (lipid, green), which coincided with each other in location.

This indicates that the UV filter and lipid components of the formulations are well mixed and that the UV filter components are located solely in the oil phase of the emulsion.

Lip skin is characterized by thin and lightly keratinized tissue with lesser amounts of lipids and no melanocyte reservoir, which makes the lip skin more vulnerable to water loss and to solar ultraviolet (UV) damage. UV exposure on skin causes oxidative stress, inflammation, and DNA damage. Furthermore, UV-induced PGE₂ has been implicated in playing a key role in the reactivation of dormant HSV and recurrence of cold sores in lip skin. Taking into account the unique needs of lip skin, a lip care composition was developed to moisturize, protect and repair the barrier function of the lips, while also to provide optimal sun protection.

Solar Ultraviolet (UV) light exposure on skin causes photoaging, sunburn, DNA damage and carcinogenesis. UVB (290-320 nm) induces erythema and DNA damage such as cyclopyrimidine dimers (CPDs) in the epidermis. UVA (320-400 nm) radiation, on the other hand, leads to oxidative stress and induces oxidative DNA damage, e.g., 8-Oxo-2′-deoxyguanosine (8-oxo-dG) and 8-hydroxy-2′-deoxyguanosine (8OHdG) in both epidermis and dermis. UV radiation (UVR) also results in inflammation, which can be measured in vitro by pro-inflammatory mediators, e.g., TNF-α, IL-8, and PGE2, and cyclooxygenase-2 (COX-2) gene expression. Furthermore, UVR could damage cells irreversibly (sunburn cells) which are eliminated by induction of apoptosis. Caspase 3, an apoptosis-related cysteine peptidase, can be used as an apoptosis biomarker to detect sunburn cells.

Sunscreen absorbs or reflects UV radiation on the skin and thus can effectively protect skin from the adverse effects of solar UVR. The protective effect of sunscreens has been extensively assessed by measuring skin erythema as expressed as sun protection factor (SPF) in humans. In vitro biological methods provide an excellent tool with which to assess the molecular damage caused by UVR and to evaluate the efficacy of topical formulations containing chemical or biological technologies in protecting skin from UVR. The in vitro reconstructed human epidermis (RHE) model has been well established as a research tool to evaluate the photoprotective effect of sunscreens and to overcome the limitations of testing on human subjects.

### Example 6

### Determining the Protective Activity Against UVB-Induced DNA Damage, Apoptosis and Inflammation Using Reconstructed Human Epidermis (EpiDerm)

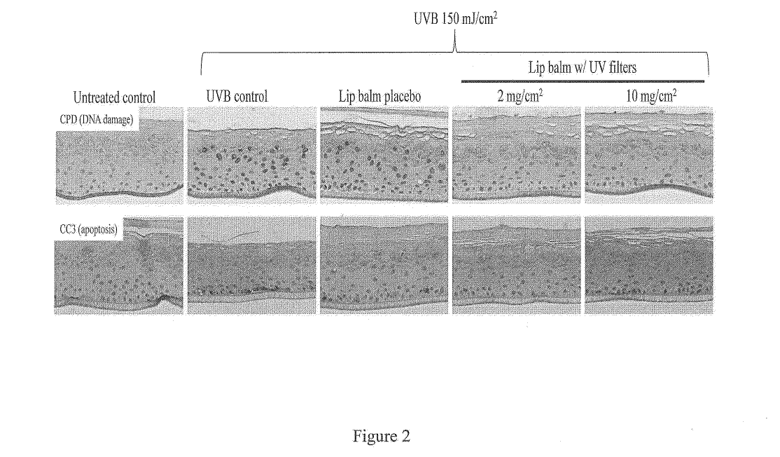

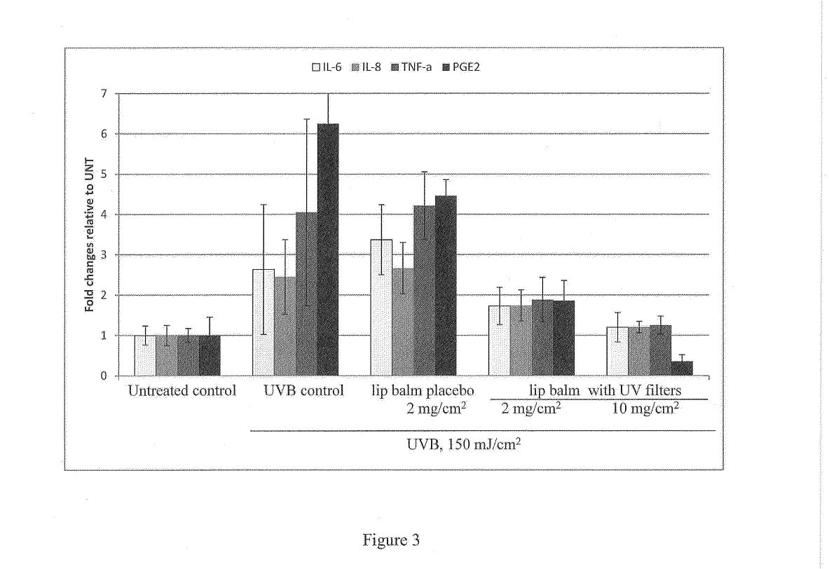

Upon receipt, reconstructed human epidermis (EpiDerm, EPI-200, made of normal human epidermal keratinocytes, MatTek, Ashland, Mass.) were placed into media (EPI-100-ASY, 1.0 ml/well of 6-well plates) and incubated overnight at 37° C./5% CO₂. The media was replenished with fresh culture media prior to study. A lip balm formulation with UV filters (formulation from Example 1A) and one without UV filters (placebo) (see Table 12) were applied topically (2 and 10 mg/cm², using positive displacement pipette tip for formulation) and then gently massaged into the skin equivalents (˜20 rotations) using the rubber side of a plunger of 1 ml syringe. Distilled H₂O served as an untreated control, and distilled H₂O plus UVB irradiation served as a UVB control. After 1 hour pre-treatment with lip balm formulations, the EpiDerm tissues were transferred to a sterile 6-well plate containing 1 ml of DPBS per well and then exposed to UVB at 150 mJ/cm². The Newport Solar Simulator System (Power unit 69920, and Lamp 91192-1000, Newport Corporate, Irvine, Calif.) was used as the UVB emitter to achieve a UVB irradiation of 150 mJ/cm². Measurement of the irradiation was taken using an ILT-1400 Handheld, Portable Radiometer/Photometer (International Light Technologies, Inc., Peabody, Mass.) with a UVB detector (SEL240/T2ACT5, 235-307 nm, International Light Technologies, Inc.). After UVB irradiation, EpiDerm tissues were transferred back to the 6-well plate containing media and incubated at 37° C./5% CO₂ for 6 hours. At the end of the incubation (6 hours post UVB irradiation), culture media were collected for the measurement of IL-6, IL-8, and TNF-α concentration by MagPix (Millipore, HCYTOMAG-60K) and PGE₂ concentration by ELISA (R&D Systems, SKGE004B). The EpiDerm tissues were harvested and placed in 10% formalin for histological processing including paraffin embedding, sectioning, immunohistological analyses of DNA damage (cyclobutane pyrimidine dimers, CPDs) and apoptosis (cleaved caspase-3, CC3). The test sample information is listed in Table 12 and photoprotective results of the lip balm with UV filters are shown in FIGS. 2 and 3.

TABLE 12
Test articles evaluated in Epiderm

| Sample No. | Test Article description |
|---|---|
| 1 | Untreated Control without UVB |
| 2 | Untreated Control with UVB (150 mJ/cm²) |
| 3 | Lip balm placebo (w/o Oxynex ST, w/o Sunfilters) |
| 4 | Lip balm with UV filters (w/Oxynex ST) |
| | (formulation of Example 1A) |

FIG. 2 illustrates that the lip balm with UV filters inhibited UVB-induced DNA damage (CPD, pink staining) and apoptosis (CC3, brown staining) in EpiDerm. As also shown in FIG. 2, UVB exposure (150 mJ/cm²) resulted in a marked increase in the numbers of cells positively stained with CPD (pink staining, DNA damage) and CC3 (brown staining, apoptosis). The lip balm with UV filters at both topical doses (2 and 10 mg/cm²) significantly reduced UVB-induced cell numbers with CPD and CC3 staining, while the lip balm without UV filters minimally inhibited UVB-induced CPD formation and CC3 positively stained cells.

FIG. 3 illustrates the lip balm with UV filters inhibiting UVB-induced pro-inflammatory mediators in EpiDerm. As shown in FIG. 3, UVB exposure (150 mJ/cm²) resulted in increases in the pro-inflammatory mediators, particularly markedly increased TNF-α and PGE₂. The lip balm placebo did not significantly reduce UVB-induced pro-inflammatory mediators. The lip balm with UV filters at both topical doses (2 and 10 mg/cm²) significantly reduced UVB-induced inflammation, with better protection at the dose of 10 mg/cm². In addition, the lip balm with UV filters significantly inhibited PGE₂ released caused by UVB.

### Example 7

### Determining the Protective Activity Against UVB-Induced DNA Damage, Apoptosis and Inflammation Using Gingival Oral Equivalents (EpiGingival)

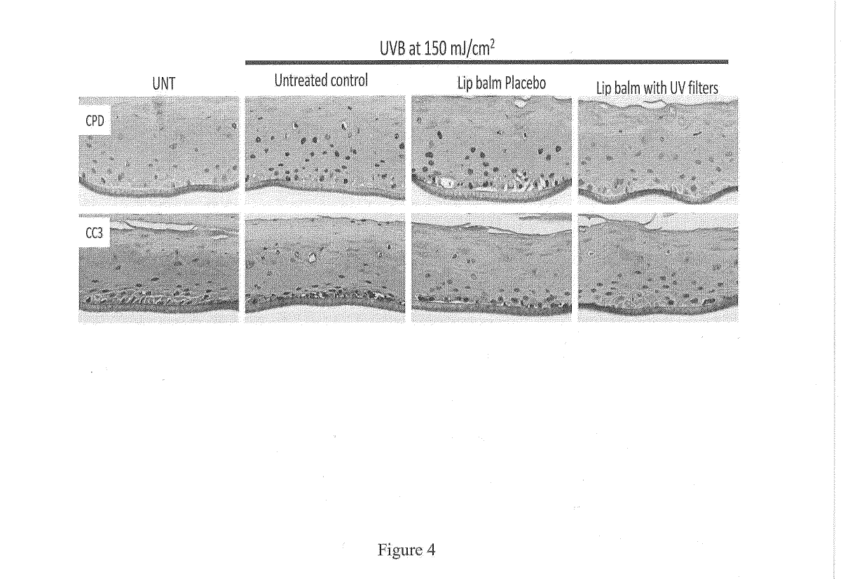

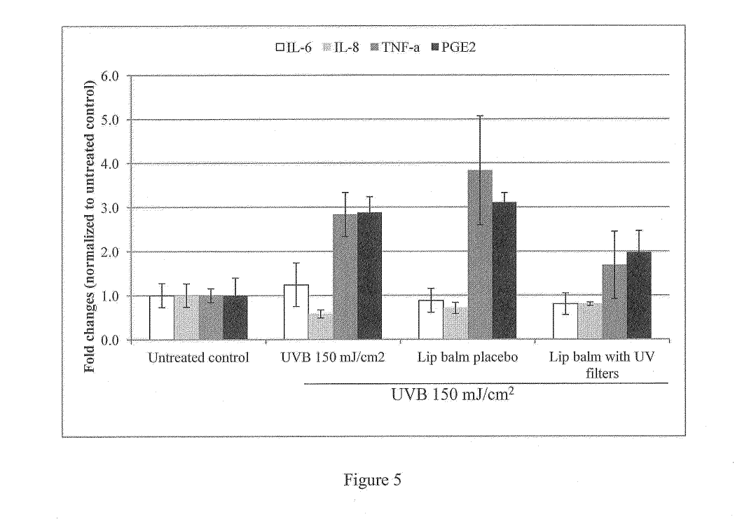

Gingival oral mucosal equivalents (EpiGingival, GIN-100, MatTek, Ashland, Mass.) use normal human oral keratinocytes (NHOK) to differentiate into tissues with a cornified, gingival phenotype. Therefore, EpiGingival might better replicate the characteristics of lip skin and was used to evaluate the photoprotective effect of lip balm formulations. Upon receipt, EpiGingival equivalents were placed into media (GIN-100-MM, 1.0 ml/well of 6-well plates) and incubated overnight at 37° C./5% CO₂. The media was replenished with fresh culture media prior to study. The lip balm with SPF (formulation of Example 1A) and without SPF (placebo) (see Table 12) were applied topically (˜4 mg/cm², using positive displacement pipette tip for formulation) and then gently massaged into the skin equivalents (˜20 rotations) using the rubber side of a plunger of 1 ml syringe. Distilled H₂O served as an untreated control, and distilled H₂O plus UVB irradiation served as a UVB control. After 1 hour pre-treatment with lip balm formulations, the EpiGingival tissues were transferred to a sterile 6-well plate containing 1 ml of DPBS per well and then exposed to UVB at 150 mJ/cm² as described in Example 6. After UVB irradiation, EpiGingival tissues were transferred back to the 6-well plate containing media and incubated at 37° C./5% CO₂ for 6 hours. At the end of the incubation (6 hours post UVB irradiation), culture media were collected for the measurement of IL-6, IL-8, and TNF-α concentration by MagPix (Millipore, HCYTOMAG-60K) and PGE₂ concentration by ELISA (R&D Systems, SKGE004B). The EpiGingival tissues were harvested and placed in 10% formalin for histological processing including paraffin embedding, sectioning, immunohistological analyses of DNA damage (cyclobutane pyrimidine dimers, CPDs) and apoptosis (cleaved caspase-3, CC3). The test sample information is listed in Table 12 and the photoprotective effect of the lip balm with UV filters is shown in FIGS. 4 and 5.

FIG. 4 illustrates that the lip balm with UV filters inhibited UVB-induced DNA damage (CPD, pink staining) and apoptosis (CC3, brown staining) in EpiGingival.

As shown in FIG. 4, UVB exposure (150 mJ/cm²) resulted in marked increases in the numbers of cells positively stained with CPD (pink staining, DNA damage) and CC3 (brown staining, apoptosis) in EpiGingival. The lip balm with UV filters significantly reduced UVB-induced cell numbers with CPD and CC3 staining, while the lip balm without UV filters minimally inhibited UVB-induced CPD formation and CC3 positively stained cells. These results are consistent with the data from EpiDerm shown in FIG. 2.

FIG. 5 illustrates that the lip balm with UV filters inhibited UVB-induced pro-inflammatory mediators in EpiGingival. As shown in FIG. 5, UVB exposure (150 mJ/cm²) resulted in markedly increased TNF-α and PGE₂. The lip balm placebo did not significantly reduce UVB-induced pro-inflammatory mediators. The lip balm with UV filters significantly reduced UVB-induced TNF-α and PGE2. Unlike EpiDerm, UVB did not induce IL-6 and IL-8 significantly.

### Example 8

### Determining the Protective Activity Against UVA-Induced DNA Damage, Apoptosis and Inflammation Using Full Thickness Skin Equivalents (EpiDerm^{FT})

EpiDerm^{FT} System consists of normal, human-derived epidermal keratinocytes and dermal fibroblasts which have been cultured to form a multilayered, highly differentiated model of the human dermis and epidermis. As used herein, EpiDerm^{FT} refers to full thickness epidermal equivalents. EpiDerm^{FT} (EFT-400, MetTek) was used for the assessment of UVA-induced skin damage and photoprotective activity of lip balm formulations. Upon receipt, EpiDerm^{FT} was placed into media (EFT-400-MM, 1.0 ml/well of 6-well plates) and incubated overnight at 37° C./5% CO₂. The media was replenished with fresh culture media prior to study. The lip balm with UV filters (formulation of Example 1A) and without UV filters (placebo) (see Table 12) were applied topically (10 mg/cm², using positive displacement pipette tip for formulation) and then gently massaged into the skin equivalents (˜20 rotations) using the rubber side of a plunger of 1 ml syringe. Distilled H₂O served as an untreated control, and distilled H₂O plus UVA irradiation served as a UVA control. After 1 hour pre-treatment with lip balm formulations, the EpiDerm^{FT} tissues were transferred to a sterile 6-well plate containing 1 ml of DPBS per well and then exposed to UVA at 30-50 J/cm² as indicated. The Newport DS-101103 UV Solar Simulator with UV-A-F filter (Sol-UV-A-F) (Newport Corporate) was used as the UVA emitter to achieve a UVA irradiation of 30-50 J/cm². Measurement of the irradiation was taken using an ILT-1400-A radiometer/photometer with a UVA probe (SSL001A, international light technologies, Inc.).

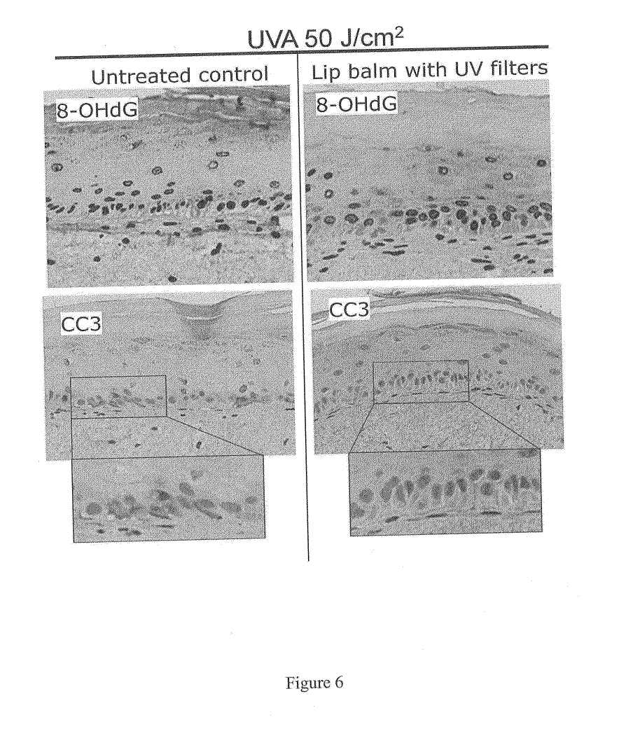

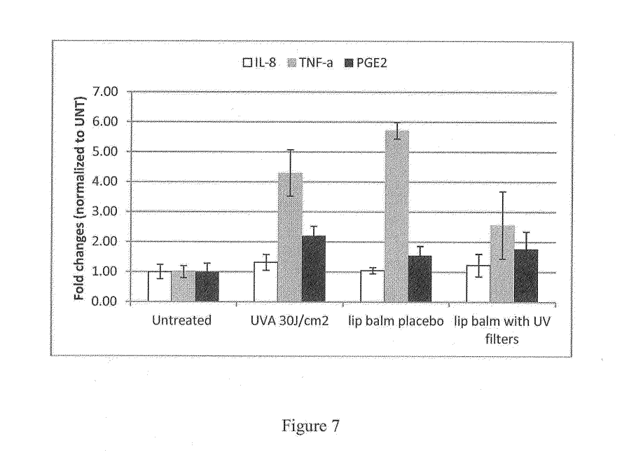

After UVA irradiation, EpiDerm^{FT} tissues were transferred back to the 6-well plate containing media and incubated at 37° C./5% CO₂ for 6 hours. At the end of the incubation (6 hours post UVA irradiation), culture media were collected for the measurement of IL-8, and TNF-α concentration by MagPix (Millipore, HCYTOMAG-60K) and PGE₂ concentration by ELISA (R&D Systems, SKGE004B). The EpiDerm^{FT} tissues were harvested and placed in 10% formalin for histological processing including paraffin embedding, sectioning, immunohistological analyses of DNA damage (8-hydroxy-2′-deoxyguanosine, 8OHdG) and apoptosis (cleaved caspase-3, CC3). The protective activities of tested lip balm formulations against UVA-induced skin damage are seen in FIGS. 6 and 7.

FIG. 6 illustrates that the lip balm with UV filters inhibited UVA-induced DNA damage and apoptosis in EpiDerm^{FT}. As shown in FIG. 6, tissues treated with UVA at 50 J/cm² have strong staining for 8-OHdG (dark brown staining, DNA damage) and CC3 (apoptosis), while the lip balm with UV filters exhibited much lower 8-OHdG staining (light brown staining), and significantly reduced CC3 staining.

FIG. 7 illustrates that the lip balm with UV filters inhibited UVA-induced pro-inflammatory mediators and PGE₂ in EpiDerm^{FT}. As shown in FIG. 7, UVA strongly induced TNF-α and PGE2, which was significantly reduced by the lip balm with UV filters, but not by the lip balm placebo.

### Example 9

### Determining the Protective Activity Against UVA-Induced Tissue Damage Using Gingival Mucosal Equivalent (EpiGingival)

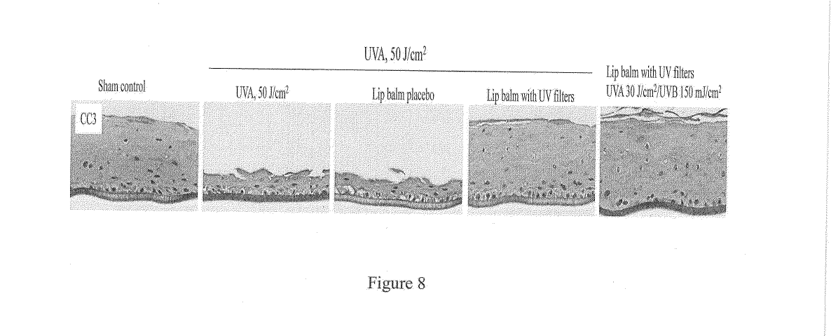

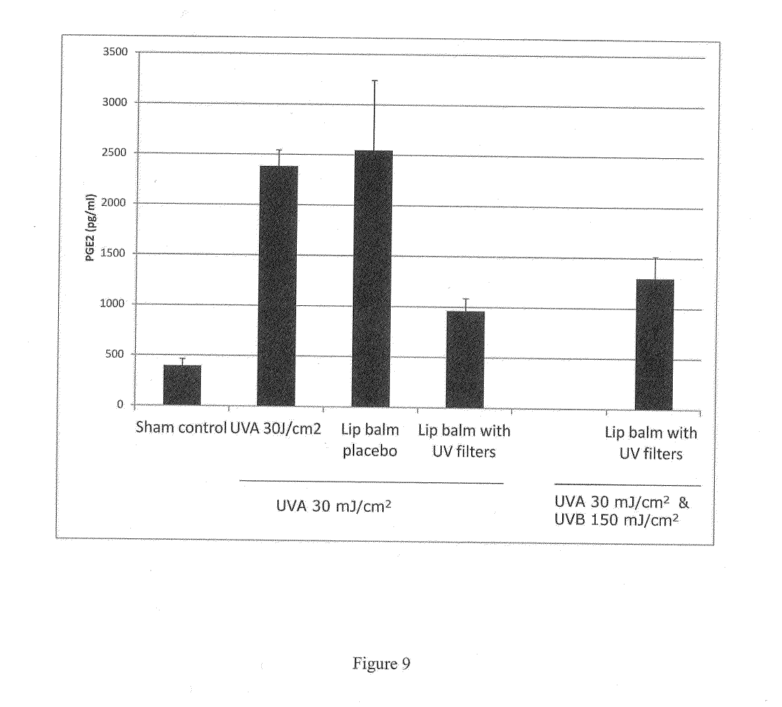

Gingival oral mucosal equivalents (EpiGingival, GIN-100, MatTek, Ashland, Mass.) were used for the assessment of protective effect of a lip balm formulation against UVA radiation. The gingival equivalents were maintained as described in Example 6. The lip balm with UV filters (formulation of Example 1A) and without UV filters (placebo) (see Table 12) were applied topically (˜4 mg/cm², using positive displacement pipette tip for formulation) and then gently massaged into the skin equivalents (˜20 rotations) using the rubber side of a plunger of 1 ml syringe. After 1 hour pre-treatment with lip balm formulations, the EpiGingival tissues were transferred to a sterile 6-well plate containing 1 ml of DPBS per well and then exposed to UVA at 30-50 J/cm² as described in Example 8. Sham irradiated EpiGingival equivalents were used as sham control. After UVA irradiation, EpiGingival equivalents were transferred back to the 6-well plate containing media and incubated at 37° C./5% CO₂ for 28 hours. At the end of the incubation (28 hours post UVA irradiation), culture media were PGE₂ concentration by ELISA (R&D Systems, SKGE004B). The EpiGingival equivalents were harvested and placed in 10% formalin for histological processing including paraffin embedding, sectioning, immunohistological analyses of DNA damage (8-hydroxy-2′-deoxyguanosine, 8OHdG) and apoptosis (cleaved caspase-3, CC3). EpiGingival equivalents were also pretreated with the lip balm with UV filters for 1 hour, then irradiated with UVA at 30 J/cm2, followed by UVB radiation at 150 mJ/cm². The protective activities of the lip balm with UV filters are shown in FIGS. 8 and 9.

As illustrated in FIG. 8, at 28 hours after UVA radiation, EpiGingival equivalents treated with UVA alone or with UVA plus lip balm placebo showed significant loss of tissue integrity, while the lip balm with UV filters protected the tissue from UVA-induced damage. Tissues pretreated with the lip balm with UV filters followed by both UVA and UVB radiations exhibited similar tissue integrity to the sham control.

FIG. 9 illustrates the significant reduction of UVA and UVB-induced PGE2 in tissues treated with the lip balm with UV filters in EpiGingival. As shown in FIG. 9, UVA at 30 J/cm² markedly increased PGE2 production, which was significantly reduced by the lip balm with UV filters, but not by the lip balm placebo. Furthermore, the lip balm with UV filters also inhibited both UVA and UVB-induced PEG2 levels.

### Example 10

### Determining the Tackiness of a Formulation

There are various methods available to measure tackiness or stickiness of a formulation. Two accepted Tack Test methods include the Probe Tack Method and the Rolling Ball Method as both defined in the Unites States Pharmacopeial Convention, Interim Revision Announcement, Nov. 1, 2013 whose disclosure is incorporated herein by reference.

A suitable Probe Tack Method uses a Malvern Kinexus Rheometer to Measure Tackiness. The basic method parameters, which may be modified as needed, are:
sample set gap between plates: 0.15 mm
compression force: 19 N
pause time after compression before pull up: 0.5 second
geometry type: 40 mm/4° rough cone with rough lower plate to model finger surfaces

### Procedure:

1. Add approximately 1.5-2 mL to the lower rheometer geometry plate. One consideration is whether or not to apply the sample to the plate as if it were applied to the skin or with minimal manipulation—i.e. the process of pumping, scooping or spreading, for example, may modify the rheological and sensorial properties of the sample.
2. Initiate rheometer pull-away method in software:
   Rheometer lowers upper cone to compress sample to 0.15 mm target gap, waits 0.5 second and then quickly pulls away from sample while measuring force vs time to generate a plot.
3. Report the area under the curve (AUC) and/or the force (Newtons) from baseline to maximum.

### Example 11

### Clinical Study

### Aims:

To establish methods to differentiate between normal and dry lips and apply them to clinically evaluate the effectiveness of a novel lip balm (described in Example 1A).

### Patients/Methods:

A photonumeric lip dryness grading scale was developed and instrumental measurement techniques (corneometer, transepidermal water loss [TEWL]) and biophysical techniques (corneocyte maturity, protein content, protease activity) validated in a non-treatment, 2-cohort (normal/dry lip) study. A randomized (1:1), evaluator-blind, parallel group (active treatment N=34/non-treatment N=33), single-centre study was conducted in females with moderate to marked lip dryness using these assessment methods.

### Results:

Compared to baseline, visual dryness at days 3 and 8 significantly improved in both groups (p=0.0007). Compared to non-treatment, the novel lip balm significantly improved visual dryness (p=0.0003), TEWL (p=0.0011) and corneometer measurements (p=0.0120) at day 8. In the active treatment group, there was no significant change in visual dryness score, TEWL and corneometer measurements after stopping product use on day 8 and day 9. Morphological differences and biomarkers indicative of enzymatic activity in lip stratum corneum did not differ between the two groups.

### Conclusions:

Objective measurements have, for the first time, been used to demonstrate that use of the novel lip balm for 7 days, significantly improves the visual appearance, barrier function and moisture content of moderately dry lips and benefits are maintained 24 hours after discontinuation of use.

All publications, including but not limited to patents and patent applications, cited in this specification are herein incorporated by reference as if each individual publication were specifically and individually indicated to be incorporated by reference herein as though fully set forth. The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A topical oil-in-water emulsion composition comprising:
a) a discontinuous oil phase;
b) a continuous aqueous phase comprising water and glycerin, wherein the glycerin is present in an amount greater than about 12% by weight, based on the total weight of the composition;
c) a thickening agent; and
d) at least one lamellar membrane structure; and
wherein the composition is a lip protectant composition.

2. The composition according to claim 1, wherein the glycerin is present in an amount from about 12% to about 40% by weight, based on the total weight of the composition.

3. The composition according to claim 1, further comprising at least one UVA sunscreen and/or UVB sunscreen.

4. The composition according to claim 3, wherein the composition comprises a UVA sunscreen and the composition has a UVA/SPF protection ratio of about 1:1.

5. The composition according to claim 4, wherein the composition comprises a UVA sunscreen which is Avobenzone.

6. The composition according to claim 5, wherein the composition further comprises a sunfilter stabilizer.

7. The composition according to claim 6, wherein the sunfilter stabilizer is diethylhexyl syringylidene malonate.

8. The composition according to claim 1, further comprising at least one dermatologically acceptable excipient selected from an antioxidant, a chelating agent, a preservative, a colorant, a sensate, a moisturizer, a humectant, a lip conditioning agent and a pH adjusting agent, and mixtures thereof.

9. The composition according to claim 1, wherein the lamellar membrane structure comprises a phospholipid, water and a lipid.

10. The composition according to claim 9, wherein the lipid is selected from at least one of an oil, a butter and a wax.

11. The composition according to claim 10, wherein the lamellar membrane structure comprises a phospholipid, water, and at least one of rice bran oil and rice bran wax; and optionally at least one of a lipid, squalane and/or squalene, a phytosterol, cholesterol or cholesterol derivative, a ceramide, or a triglyceride.

12. The composition according to claim 9, wherein the lamellar membrane structure further comprises a polyvalent alcohol.

13. The composition according to claim 1, wherein the composition further comprises a pharmaceutically active agent.

14. The composition according to claim 8, wherein the preservative is a combination of capryloyl glycine and a glycol.

15. The composition according to claim 1, wherein the lip protectant composition is a lip balm, a lip cream, or a stick lip balm.

16. A method for moisturizing, and protecting, repairing, or restoring the skin lipid barrier of the lips of a mammal, the method comprising applying to the lips of the mammal in need thereof a therapeutically effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent; and
(d) at least one lamellar membrane structure; and
wherein the composition is a lip protectant composition.

17. The method according to claim 16, wherein the emulsion composition further comprises at least one dermatologically acceptable excipient selected from an antioxidant, a chelating agent, a preservative, a colorant, a sensate, a moisturizer, a humectant, a lip conditioning agent and a pH adjusting agent, and mixtures thereof.

18. The method according to claim 16, wherein the lamellar membrane structure comprises a phospholipid, a lipid, and water.

19. The method according to claim 18, wherein the lamellar membrane structure further comprises a polyvalent alcohol.

20. The method according to claim 18, wherein the lipid is at least one of rice bran oil and rice bran wax.

21. A topical oil-in-water emulsion composition comprising:
(a) a discontinuous oil phase;
(b) a continuous aqueous phase;
(c) a thickening agent;
(d) at least one lamellar membrane structure, comprising a phospholipid, water, and at least one of rice bran oil and rice bran wax; and
(e) optionally at least one dermatologically acceptable excipient.

22. The composition according to claim 21, wherein the discontinuous oil phase comprises at least one of rice bran oil and rice bran wax.

23. The composition according to claim 21, further comprising at least one dermatologically acceptable excipient selected from an antioxidant, a chelating agent, a preservative, a colorant, a sensate, a moisturizer, a humectant, a lip conditioning agent and a pH adjusting agent, and mixtures thereof.

24. A lamellar membrane structure concentrate composition which comprises at least one lamellar membrane structure, comprising a phospholipid, water, and at least one of rice bran oil and rice bran wax; and optionally at least one of a lipid, squalane and/or squalene, a phytosterol, cholesterol or cholesterol derivative, a ceramide, or a triglyceride.

25. The lamellar membrane structure concentrate composition according to claim 24 wherein said phytosterol is obtained from shea butter, the triglyceride is caprylic/capric triglyceride, the squalane is obtained from olive oil, the phospholipid is hydrogenated lecithin, and the ceramide is ceramide-3.

26. A method of protecting the lips of a mammal with broad spectrum protection of a UVA sunscreen and a UVB sunscreen, and enriched in UVA protection, the method comprising applying to the lips of the mammal in need thereof an effective amount of a topical oil-in-water emulsion composition comprising:
(a) a discontinuous oil phase;
(b) a continuous aqueous phase comprising water and glycerin, wherein the glycerin is present in an amount greater than about 12% by weight, based on the total weight of the composition;
(c) a thickening agent;
(d) at least one lamellar membrane structure; and
(e) at least one UVA sunscreen and at least one UVB sunscreen; and wherein the UVA/SPF protection ratio is about 1:1; and
wherein the composition is a lip protectant composition.

27. The method according to claim 26, wherein the enriched UVA sunscreen in the composition protects against the photodegradation of pheomelanin in the lips.
